(19) United States
(12) Patent Application Publication
(10) Pub. No.: US 2022/0257491 A1
(43) Pub. Date: Aug. 18, 2022

(54) Prebiotic Oral Care Compositions and Methods

(71) Applicants: Colgate-Palmolive Company, New York, NY (US); Katholieke Universiteit Leuven, Leuven (BE); Universiteit Gent, Gent (BE)

(72) Inventors: Wim TEUGHELS, Everberg (BE); Tim VERSPECHT, Leuven (BE); Kristel BERNAERTS, Leuven (BE); Marc QUIRYNEN, Leuven (BE); Nico BOON, Gent (BE); Carlo DAEP, Brooklyn, NY (US); James MASTERS, Ringoes, NJ (US)

(21) Appl. No.: 17/673,480

(22) Filed: Feb. 16, 2022

Related U.S. Application Data
(60) Provisional application No. 63/150,257, filed on Feb. 17, 2021.

(51) Int. Cl. A61K 8/60; A61K 8/25; A61K 8/29; A61K 8/34; A61K 8/21; A61Q 11/00

(52) U.S. Cl. CPC A61K 8/60; A61K 8/25; A61K 8/29; A61K 2800/28; A61K 8/21; A61Q 11/00; A61K 8/345

(57) ABSTRACT

The disclosure relates to methods of enhancing beneficial oral bacteria and decreasing harmful oral bacteria comprising administering oral care compositions comprising a saccharide prebiotic, e.g., selected from D-mannose, N-acetyl-D-mannosamine and mixtures thereof; and oral care compositions for use in such methods. The disclosure also relates to methods of using prebiotic oral care compositions, methods of screening, and methods of manufacture.

### FIELD

The field relates to compositions and methods of enhancing beneficial oral bacteria and decreasing harmful oral bacteria comprising administering oral care compositions comprising a saccharide prebiotic, wherein the saccharide prebiotic comprises mannose (e.g., D-(+)-mannose) and/or N-acetyl-D-mannosamine (NADM); and oral care compositions for use in such methods. The field also relates to methods of using prebiotic oral care compositions, methods of screening, and methods of manufacture.

### BACKGROUND

Different types of sugar are present in our diet and come into contact with plaque during eating. The breakdown of sugars is an important step that influences the plaque environment. Sugar metabolism requires specific enzymes. The genetic disposition and expression of pathway dictates which strains are able to grow on which type of sugars.

The occurrence of high amount of certain sugar may provide a selection advantage to certain species over others, simply due to the fact that they are able to grow on the metabolite but also due to effects that influence the environment such as acid production, bacteriocins, and/or breakdown products that may be metabolized by further species.

When there is an increase in the intake of certain fermentable carbohydrates, this may cause pH to drop in a user's oral cavity. Not only does the acid damage the teeth, but the acidic environment causes a shift toward a more aciduric and acidogenic bacterial, and certain cariogenic bacteria, which are typically found in relatively small amounts, may actually increase in number and size. Ultimately, this can lead to dental caries. Some species of oral pathogenic bacteria (e.g. Porphyromonas gingivalis, Tannerella forsythia and Aggregatibacter actinomycetemcomitans) have been implicated in the development of periodontal diseases such as periodontitis, gingivitis, necrotizing periodontitis, necrotizing gingivitis and peri-implantitis. Certain species of oral pathogenic bacteria have been implicated in tooth decay (e.g. Streptococcus mutans). Current strategies to address these problems include the use of oral care products containing broad-spectrum antibacterial agents. Such product, however, can inhibit or kill bacteria irrespective of whether the bacteria are beneficial or detrimental. Moreover, pathogens may evolve to develop resistance to antimicrobial agents. Accordingly, alternative methods of prophylaxis and treatment are needed.

“Probiotics” are microorganisms that provide health benefits when consumed. “Prebiotics” are ingestible ingredients that allow specific changes, both in the composition and/or activity in the gastrointestinal microflora that confer benefits upon the host well-being and health. While prebiotics are generally known for influencing the composition of the gastrointestinal microflora, there has been little attention directed to using a similar prebiotic strategy to encourage beneficial oral bacteria. Rather than trying to stimulate beneficial bacteria in the mouth, the emphasis has been on avoiding and promptly removing compounds, like sucrose, that encourage harmful oral bacteria as well as using antibacterial agents to reduce oral plaque.

The use of prebiotics for maintaining and/or restoring the health-associated homeostasis of the oral microbiota and modulation of the host response is being investigated more. However, there is currently a need for the stimulation of beneficial/commensal bacteria through potentially prebiotic substrates, resulting in more host-compatible biofilms that harbor lower amounts of pathogens, show decreased virulence and have less inflammatory potential. It would be desirable to identify new substrates, for example, which can stimulate the beneficial/commensal oral bacteria in terms of growth and/or metabolism and by consequence inhibit pathogenic oral bacteria, decrease virulence gene expression and reduce the inflammatory response of oral keratinocytes exposed to substrate-treated biofilms.

### SUMMARY OF THE INVENTION

The invention contemplates that certain in vitro multi-species oral biofilms can surprisingly be modulated by stimulating certain beneficial/commensal bacteria with potentially prebiotic substrates, e.g., saccharide prebiotics. This stimulation can result in more host-compatible biofilms that comprise lower amounts of pathogens, demonstrate decreased virulence and have less inflammatory potential as measured by certain inflammatory biomarkers.

In one aspect, the substrates (e.g., saccharide prebiotics), can stimulate certain beneficial/commensal oral bacteria in terms of growth and/or metabolism. In yet another aspect, by stimulating certain beneficial/commensal oral bacteria also results in the inhibition of certain pathogenic oral bacteria, decrease virulence gene expression and reduce the inflammatory response of oral keratinocytes exposed to multi-species oral biofilms that are treated with these substrates. Here, without being bound by theory, the inventors have surprisingly found that both the presence/absence and the orientation of an N-acetyl group on certain prebiotic substrates, e.g., D-mannose and N-acetyl-D-mannosamine can have a role in certain aspects when considering compositional, metabolic and virulence changes. Without being bound by theory, the compounds may possibly help facilitate the transacetylation of COX1 and COX2 to stop the production of prostaglandins. In turn, mediating prostaglandin levels may potentially help mitigate the inflammatory response at local soft tissue sites in the oral cavity. In certain aspects, and without being bound by theory, this may generally exist only at a substrate concentration of about 1 M and may often be dependent on the biofilm aspect under consideration.

Oral care compositions described herein comprising a saccharide prebiotic identified in this manner, e.g., mannose (e.g., D-mannose) (e.g., D-(+)-mannose) and/or N-acetyl-D-mannosamine (NADM), in certain aspects, can increase the growth of certain beneficial/commensal bacteria in the oral microbiota. Such beneficial/commensal bacteria include, e.g: A. naeslundii; A. viscosus; S. gordonii: S. mitis; S. oralis; S. salivarius; S. sanguinis; V. parvula. In some aspects these selected saccharide prebiotics that encourage the growth of beneficial bacteria also negatively affect the growth of certain pathogenic strains of bacteria. In certain aspects these pathogenic strains include, e.g.: A. actinomycetemcomitans; F. nucleatum; P. gingivalis; P. intermedia; S. sobrinus; and S. mutans.

The present invention contemplates that selective stimulation of beneficial bacteria provides a valid preventive/prophylaxis approach for oral health. Without being bound by any theory, it is thought that since bacteria need certain substrates in order to grow, one can obtain certain microbiological shifts in the bacterial environment by selectively encouraging the growth of an individual's beneficial endogenous bacterial population by providing them with appropriate substrates. For example, without being bound by theory, select substrates are preferentially utilized by certain microorganisms. By selecting the appropriate substrate, it is possible encourage the growth of certain microorganisms (e.g., beneficial endogenous bacterial strains) while also directly or indirectly suppressing the growth of select other microorganisms (endogenous pathogenic bacterial strains).

In one aspect, the invention relates to a novel prebiotic approach that selectively promotes the growth of beneficial endogenous bacteria but not the growth of harmful bacteria by using an oral care composition comprising a prebiotically effective amount of a saccharide prebiotic, e.g., a saccharide prebiotic selected from mannose (e.g., D-(+)-mannose), N-acetyl-D-mannosamine and combinations thereof. For example, this may include use of compositions which promote the growth of at least one of the above-listed beneficial bacteria while not simultaneously promoting growth of any of the above-listed harmful bacteria.

An oral care composition (Composition 1) useful in the methods of the present invention is an oral care composition comprising an effective amount of at least one saccharide prebiotic, e.g., a saccharide prebiotic comprising a substrate selected from D-mannose (e.g., D-(+)-mannose), N-acetyl-D-mannosamine (NADM), and combinations thereof e.g., in an amount effective to promote the growth of beneficial endogenous bacteria in the oral cavity and inhibit pathogenic oral bacteria (e.g., decrease virulence gene expression and reduce the inflammatory response of oral keratinocytes). For example, in various aspects the oral care compositions useful in the methods of the present invention include:
1.1 Composition 1, wherein the saccharide prebiotic is a monosaccharide, e.g., mannose (e.g., D-mannose) (e.g., D-(+)-mannose), e.g., a hexosamine mono saccharide (e.g., N-acetyl-D-mannosamine).
1.2 Composition 1 or 1.1, wherein the saccharide prebiotic comprises mannose (e.g., D-mannose) (e.g., D-(+)-mannose).
1.3 Composition 1, 1.1, or 1.2, wherein the saccharide prebiotic comprises N-acetyl-D-mannosamine.
1.4 Any of the preceding compositions, wherein the saccharide prebiotic further comprises, N-acetyl-D-glucosamine, or D-lactose (e.g., α-D-lactose), or D-raffinose (e.g., D-(+)-raffinose), or D-trehalose (e.g., D-(+)-trehalose), or combinations thereof.
1.5 Any foregoing composition wherein the amount of saccharide prebiotic is at least 0.1%, e.g., 0.1% to 5%, e.g., about 0.5%, 1% or 2% by weight of the composition (e.g., or from 0.5%-5% w/v) (e.g., or from 0.5%-2% w/v) (e.g., or about 1% w/v).
1.6 Any foregoing composition wherein the amount of saccharide prebiotic is from 0.5 μmon to 10 mmol/L, from 0.5 μmon to 5 mmol/L, from 1 μmon to 5 mmol/L, from 5 μmon to 10 mmol/L, from 0.75 μmon to 1.5 mmol/L, about 0.75 mmol/L, about 1 mmol/L, about 1.5 mmol/L or about 1.75 mmol/L.
1.7 Any foregoing composition wherein the saccharide prebiotic is not derived from a plant extract.
1.8 Any foregoing composition wherein the composition promotes the growth or expression in the oral cavity of one or more beneficial or commensal endogenous bacterial species, wherein said species are one or more selected from the group consisting of: A. naeslundii; A. viscosus; S. gordonii: S. mitis; S. oralis; S. salivarius; S. sanguinis; V. parvula.
1.9 Any foregoing composition, wherein the composition negatively affects the growth or expression in the oral cavity of one or more pathogenic bacterial species, wherein said species are one or more selected from the group consisting of: A. actinomycetemcomitans; F. nucleatum; P. gingivalis; P. intermedia; S. Sobrinus; and S. mutans.
1.10 The compositions of 1.9, wherein the composition negatively affects the growth in the oral cavity of one or more pathogenic bacterial species, wherein said species are one or more selected from the group consisting of A. actinomycetemcomitans; F. nucleatum; P. gingivalis; P. intermedia.
1.11 Any foregoing composition wherein the composition further comprises at least one species of bacteria that has beneficial effects on oral health.
1.12 Composition 1.11 wherein the species of bacteria that has beneficial effects on oral health is selected from A. naeslundii; A. viscosus; S. gordonii: S. mitis; S. oralis; S. salivarius; S. sanguinis; V. parvula and combinations thereof.
1.13 Any of the preceding compositions further comprising an anti-calculus agent.
1.14 Any of the preceding compositions further comprising an anti-calculus agent which is a polyphosphate, e.g., pyrophosphate, tripolyphosphate, or hexametaphosphate, e.g., in sodium salt form.
1.15 Any of the preceding compositions comprising at least one surfactant selected from sodium lauryl sulfate, cocamidopropyl betaine, and combinations thereof.
1.16 Any of the preceding compositions comprising an anionic surfactant, e.g., selected from sodium lauryl sulfate, sodium laureth sulfate, and mixtures thereof.
1.17 Any of the preceding compositions comprising sodium lauryl sulfate, in an amount from 0.5-3% by wt of the composition.
1.18 Any of the preceding compositions comprising at least one humectant.
1.19 Any of the preceding compositions comprising at least one humectant selected from glycerin, sorbitol and combinations thereof.
1.20 Any of the preceding compositions comprising at least one polymer.
1.21 Any of the preceding compositions comprising at least one polymer selected from polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum), and combinations thereof.
1.22 Any of the preceding compositions comprising one or more abrasives, e.g., silica, calcium carbonate, or calcium phosphate abrasives.
1.23 Any of the preceding compositions comprising gum strips or fragments. 1.24 Any of the preceding compositions comprising flavoring, fragrance and/or coloring.
1.25 Any composition obtained or obtainable by combining the ingredients as set forth in any of the preceding compositions.
1.26 Any of the preceding oral care compositions, wherein the composition is a mouthwash, toothpaste, tooth gel, tooth powder, non-abrasive gel, mousse, foam, mouth spray, lozenge, oral tablet, or dental implement.
1.27 Any of the preceding compositions wherein the composition is a toothpaste or a mouthwash.
1.28 Any of the preceding compositions wherein the composition is a toothpaste optionally further comprising one or more of one or more of water, abrasives, surfactants, foaming agents, vitamins, polymers, enzymes, humectants, thickeners, preservatives, flavorings, colorings and/or combinations thereof.
1.29 Any preceding composition, wherein the composition is a toothpaste further comprising water, abrasive, surfactant, humectant, thickener, and flavoring.
1.30 Any preceding composition wherein the composition is a toothpaste obtained or obtainable by a method of mixing with a toothpaste base, e.g., a toothpaste base comprising one or more of one or more of water, abrasives, surfactants, foaming agents, vitamins, polymers, enzymes, humectants, thickeners, antimicrobial agents, preservatives, flavorings, colorings and/or combinations thereof.
1.31 Any preceding composition for use in selectively promoting, in an oral cavity: growth, metabolic activity or colonization of bacteria that have beneficial effects on oral health, relative to growth, metabolic activity or colonization of pathogenic oral bacteria.
1.32 Composition 1.31 wherein the bacteria that have beneficial effects on oral health are selected from A. naeslundii; A. viscosus; S. gordonii: S. mitis; S. oralis; S. salivarius; S. sanguinis; V. parvula and combinations thereof.
1.33 Any of compositions 1.31 to 1.32 wherein the pathogenic oral bacteria are selected from A. actinomycetemcomitans; F. nucleatum; P. gingivalis; P. intermedia; S. mutans; S. Sobrinus and combinations thereof.
1.34 Composition 1.33, wherein the pathogenic oral bacteria are selected from A. actinomycetemcomitans; F. nucleatum; P. gingivalis; P. intermedia, and combinations thereof.
1.35 Any of compositions 1.31 to 1.34, wherein the composition selectively promotes growth, metabolic activity or colonization of bacteria that have beneficial effects on oral health, relative to growth, metabolic activity or colonization of pathogenic oral bacteria, after 24 hours incubation with the bacteria that have beneficial effects on oral health and the pathogenic oral bacteria.
1.36 Any of compositions 1.31 to 1.34, wherein the composition selectively promotes growth, metabolic activity or colonization of bacteria that have beneficial effects on oral health, relative to growth, metabolic activity or colonization of pathogenic oral bacteria, after 48 hours incubation with the bacteria that have beneficial effects on oral health and the pathogenic oral bacteria.
1.37 Any preceding composition for use in selectively promoting, in an oral cavity, biofilm formation by bacteria that have beneficial effects on oral health, relative to biofilm formation by pathogenic oral bacteria.
1.38 Composition 1.36 wherein the bacteria that have beneficial effects on oral health are selected from A. naeslundii; A. viscosus; S. gordonii: S. mitis; S. oralis; S. salivarius; S. sanguinis; V. parvula, and combinations thereof.
1.39 Any of compositions 1.36 or 1.37 wherein the pathogenic oral bacteria are selected from A. actinomycetemcomitans; F. nucleatum; P. gingivalis; P. intermedia; S. mutans; S. Sobrinus, and combinations thereof.
1.40 Composition 1.39, wherein the pathogenic bacteria is selected from: A. actinomycetemcomitans; F. nucleatum; P. gingivalis; P. intermedia, and combinations thereof.
1.41 Any of the preceding compositions, wherein the prebiotic substrate can decrease virulence gene expression of one or more of the following genes in A. actinomycetemcomitans: aae, emaA, paH, cdtB, ltxA, omp100, orf859, vapA and flp.
1.42 The composition of 1.41, wherein the prebiotic substrate can decrease gene expression of orf859.
1.43 Any of the preceding compositions, wherein the prebiotic substrate can decrease virulence gene expression of one or more of the following genes in P. gingivalis: fim A, kgp, partC, rgpA.
1.44 The composition of 1.43, wherein the prebiotic substrate can decrease gene expression of rgpA, kgp, and partC.
1.45 Any of the preceding compositions, wherein the prebiotic substrate can decrease virulence gene expression of one or more of the following genes in P. intermedia: adpc, clpB, DnaK, DnaJ, ECF, GroES, HtpG, KpsD, inpA, phg.
1.46 The composition of 1.45, wherein the prebiotic substrate can decrease gene expression of clpB, dnaJ HtpG, KpsD, DnaK, GroES, inpA and phg.
1.47 Any of the preceding compositions, wherein the prebiotic substrate can decrease virulence gene expression of one or more of the following genes in F. nucleatum: ABC transporter permease, heroin receptor, EF-G.
1.48 Any of the preceding compositions, wherein the prebiotic substrate can decrease the absolute number (e.g., measured via qPCR) of one or more pathogenic bacteria selected from: A. actinomycetemcomitans; F. nucleatum; P. gingivalis; P. intermedia, and combinations thereof.
1.49 Any of the preceding compositions, wherein treatment comprises administration with a saccharide prebiotic (e.g., D-mannose) (e.g., D-(+)-mannose) and/or N-acetyl-D-mannosamine (NADM)).
1.50 Any of the preceding compositions, wherein treatment results in an oral microbiota with greater than 60% beneficial/commensal bacteria (e.g., from 60%-98% beneficial/commensal bacteria (Geq/mL)) (e.g., from 90%-98% beneficial/commensal bacteria (Geq/mL).
1.51 Any of the preceding compositions, wherein treatment with a saccharide prebiotic comprising mannose (e.g., D-mannose) (e.g., D-(+)-mannose) and/or N-acetyl-D-mannosamine (NADM)) results in an oral microbiota with less than 40% periodontal pathogenic bacterial species (e.g., from 1.25%-39% periodontal pathogenic bacterial species (Geq/mL)) (e.g., from 25%-39% periodontal pathogenic bacterial species (Geq/mL)) (e.g., from 1.25%-2.5% periodontal pathogenic bacterial species (Geq/mL)).
1.52 Any of the preceding compositions, wherein treatment with a saccharide prebiotic comprising mannose ((e.g., D-mannose) (e.g., D-(+)-mannose) and/or N-acetyl-D-mannosamine (NADM)) can decrease the inflammatory response in oral keratinocytes.
1.53 The composition of 1.52, wherein the saccharide prebiotic can decrease the gene expression of inflammatory biomarker in oral keratinocytes, wherein the biomarker is selected from: IL-1β, IL-6, IL-8, MMP-8, TNF-α, and combinations thereof.
1.54 Any of the preceding compositions, wherein the saccharide prebiotic can decrease organic acid production in biofilm.
1.55 Composition 1.54, wherein the organic acid is selected from lactate, formate, acetate, propionate, butyrate, and combinations thereof.

For example, the invention provides in one embodiment, an oral care composition comprising an effective amount of at least one saccharide prebiotic, e.g., a saccharide prebiotic comprising mannose ((e.g., D-mannose) (e.g., D-(+)-mannose) and/or N-acetyl-D-mannosamine (NADM), e.g., any of Compositions 1, et seq., for use in selectively promoting, in an oral cavity: growth, metabolic activity or colonization of bacteria that have beneficial effects on oral health, relative to growth, metabolic activity or colonization of pathogenic oral bacteria.

For example, the invention provides in another embodiment, an oral care composition comprising an effective amount of at least one saccharide prebiotic, e.g., a saccharide comprising mannose ((e.g., D-mannose) (e.g., D-(+)-mannose) and/or N-acetyl-D-mannosamine (NADM)), e.g., any of Compositions 1, et seq., for use in selectively promoting, in an oral cavity, biofilm formation by bacteria that have beneficial effects on oral health, relative to biofilm formation by pathogenic oral bacteria.

For example, the invention provides in another embodiment, an oral care composition comprising an effective amount of at least one saccharide prebiotic, e.g., a saccharide prebiotic comprising mannose ((e.g., D-mannose) (e.g., D-(+)-mannose) and/or N-acetyl-D-mannosamine (NADM)), e.g., any of Compositions 1, et seq., for use in maintaining and/or re-establishing a healthy oral microbiota.

For example, the invention provides in another embodiment, an oral care composition comprising an effective amount of at least one saccharide prebiotic, e.g., a saccharide prebiotic comprising mannose ((e.g., D-mannose) (e.g., D-(+)-mannose) and/or N-acetyl-D-mannosamine (NADM)), e.g., any of Compositions 1, et seq. for use in treating or preventing one or more of gingivitis, periodontitis, peri-implantitis, peri-implant mucositis, necrotizing gingivitis, necrotizing periodontitis, systemic health disorders and caries.

Further provided is a method for prophylaxis or reduction of tooth decay, caries and/or gum disease, comprising contacting the oral cavity with a composition comprising an effective amount of at least one saccharide prebiotic, e.g., a saccharide prebiotic comprising mannose ((e.g., D-mannose) (e.g., D-(+)-mannose) and/or N-acetyl-D-mannosamine (NADM)), e.g., any of Compositions 1, et seq., e.g by brushing, e.g. on a regular basis over a sufficient period of time to enhance the growth of beneficial bacteria in the oral cavity.

Further provided is a method for increasing the amount of beneficial endogenous bacteria in the oral cavity of a subject in need thereof comprising administering to a subject in need thereof an oral care composition comprising an effective amount of at least one saccharide prebiotic, e.g., a saccharide prebiotic comprising mannose ((e.g., D-mannose) (e.g., D-(+)-mannose) and/or N-acetyl-D-mannosamine (NADM)), e.g., any of Compositions 1, et seq., e.g., wherein the amount of saccharide prebiotic in the composition promotes the growth of beneficial endogenous bacteria, e.g., wherein the beneficial endogenous bacteria are one or more species selected from the group consisting of A. naeslundii; A. viscosus; S. gordonii: S. mitis; S. oralis; S. salivarius; S. sanguinis; V. parvula.

Further provided is a method of selectively promoting, in an oral cavity of a subject: growth, metabolic activity or colonization of bacteria that have beneficial effects on oral health, relative to growth, metabolic activity or colonization of pathogenic oral bacteria; the method comprising contacting the oral cavity with an oral care composition an effective amount of at least one saccharide prebiotic, e.g., a saccharide prebiotic comprising mannose ((e.g., D-mannose) (e.g., D-(+)-mannose) and/or N-acetyl-D-mannosamine (NADM)), e.g., any of Compositions 1, et seq.

Further provided is a method of selectively promoting, in an oral cavity of a subject, biofilm formation by bacteria that have beneficial effects on oral health, relative to biofilm formation by pathogenic oral bacteria; the method comprising contacting the oral cavity with an oral care composition comprising an effective amount of at least one saccharide prebiotic, e.g., a saccharide prebiotic comprising mannose ((e.g., D-mannose) (e.g., D-(+)-mannose) and/or N-acetyl-D-mannosamine (NADM)), e.g., any of Compositions 1, et seq.

Further provided is method for decreasing the amount of pathological endogenous bacteria in the oral cavity of a subject in need thereof comprising administering to a subject an oral care composition comprising an effective amount of at least one saccharide prebiotic, e.g., a saccharide prebiotic comprising ((e.g., D-mannose) (e.g., D-(+)-mannose) and/or N-acetyl-D-mannosamine (NADM)), e.g., any of Compositions 1, et seq., e.g., wherein the amount of the saccharide prebiotic in the composition inhibits the growth of pathological endogenous bacteria, e.g., wherein the pathological endogenous bacteria are one or more species selected from the group consisting of: A. actinomycetemcomitans; F. nucleatum; P. gingivalis; P. intermedia; S. Sobrinus; S. mutans.

Further provided is a method of maintaining and/or re-establishing a healthy oral microbiota in a subject, the method comprising contacting an oral cavity of the subject with an oral care composition comprising an effective amount of at least one saccharide prebiotic, e.g., a saccharide prebiotic comprising mannose ((e.g., D-mannose) (e.g., D-(+)-mannose) and/or N-acetyl-D-mannosamine (NADM)), e.g., any of Compositions 1, et seq.

Further provided is a method of preventing or mitigating or treating one or more of gingivitis, periodontitis, peri-implantitis, peri-implant mucositis, necrotizing gingivitis, necrotizing periodontitis, systemic health disorders and caries in a subject, by selectively promoting, in an oral cavity of a subject: growth, metabolic activity or colonization of bacteria that have beneficial effects on oral health, relative to growth, metabolic activity or colonization of pathogenic oral bacteria, the method comprising contacting an oral cavity of the subject with an oral care composition comprising an effective amount of at least one saccharide prebiotic, e.g., a saccharide prebiotic comprising mannose (e.g., D-mannose) (e.g., D-(+)-mannose) and/or N-acetyl-D-mannosamine (NADM)), e.g., any of Compositions 1, et seq.

Further provided is a use of a saccharide prebiotic, e.g., a saccharide prebiotic comprising mannose ((e.g., D-mannose) (e.g., D-(+)-mannose) and/or N-acetyl-D-mannosamine (NADM)), e.g., in any of Compositions 1, et seq., for prophylaxis or reduction of tooth decay, caries and/or gum disease, or to enhance the growth of beneficial bacteria in the oral cavity, e.g., by contacting the dental surface with a an effective amount of at least one saccharide prebiotic, e.g., a saccharide prebiotic comprising mannose ((e.g., D-mannose) (e.g., D-(+)-mannose) and/or N-acetyl-D-mannosamine (NADM)), e.g., any of Compositions 1, et seq.

Further provided is a use of a saccharide prebiotic, e.g., a saccharide prebiotic comprising mannose ((e.g., D-mannose) (e.g., D-(+)-mannose) and/or N-acetyl-D-mannosamine (NADM)), in the manufacture of an oral care composition, e.g., any of Compositions 1, et seq., for prophylaxis or reduction of tooth decay, caries and/or gum disease, or to enhance the growth of beneficial bacteria in the oral cavity.

In still another aspect, the invention relates to the use of a saccharide prebiotic, e.g., a saccharide prebiotic comprising mannose ((e.g., D-mannose) (e.g., D-(+)-mannose) and/or N-acetyl-D-mannosamine (NADM)), in the manufacture of an oral care product, e.g., any of Compositions 1, et seq., to promote growth of beneficial endogenous bacteria, but not the growth of harmful bacteria.

Further provided is the use of an oral care composition (e.g., any of Composition 1, et seq.) of a saccharide prebiotic, e.g., a saccharide prebiotic comprising ((e.g., D-mannose) (e.g., D-(+)-mannose) and/or N-acetyl-D-mannosamine (NADM)), to:
(a) selectively promote growth, metabolic activity or colonization of bacteria that have beneficial effects on oral health, relative to growth, metabolic activity or colonization of pathogenic oral bacteria;
(b) selectively promote biofilm formation by bacteria that have beneficial effects on oral health, relative to biofilm formation by pathogenic oral bacteria;
(c) maintain and/or re-establish a healthy oral microbiota in a subject; or (d) prevent one or more of gingivitis, periodontitis, peri-implantitis, peri-implant mucositis, necrotizing gingivitis, necrotizing periodontitis and caries in a subject.

In another aspect, the invention relates to methods of screening for compounds that promote the growth of beneficial oral bacteria, wherein screening steps include:
Determining the ability of a first compound (e.g., test compound) to promote the growth of beneficial oral bacteria, while simultaneously negatively affecting the growth of pathogenic oral bacteria, e.g., comparing growth of at least one species of beneficial oral bacteria and at least one species of pathogenic oral bacteria, e.g., wherein effect of the first compound on growth is measured by optical density or biofilm formation following at least 24 hours culture in the presence and absence of the first compound;
Optionally determining the ability of a second compound (e.g., control compound) to promote the growth of beneficial bacteria, while simultaneously negatively affecting the growth of pathogenic oral bacteria;
Optionally comparing the profile of the first compound with the profile of the second compound;
Selecting a test compound for further testing based upon its ability to promote the growth of beneficial oral bacteria and inhibit the growth of pathogenic oral bacteria, e.g., as compared to the control compound.
   For example, the control compound in the foregoing method of screening may be a saccharide prebiotic, e.g., a saccharide comprising ((e.g., D-mannose) (e.g., D-(+)-mannose) and/or N-acetyl-D-mannosamine (NADM)), e.g., any of Compositions 1, et seq. In some embodiments, the beneficial oral bacteria are one or more species selected from the group consisting of A. naeslundii; A. viscosus; S. gordonii: S. mitis; S. oralis; S. salivarius; S. sanguinis; V. parvula. In some embodiments, the pathogenic oral bacteria are one or more species selected from the group consisting of: A. actinomycetemcomitans; F. nucleatum; P. gingivalis; P. intermedia; S. Sobrinus; and S. mutans. The invention further provides the use of a compound identified in such a screening method in any of the foregoing methods and uses.

### DETAILED DESCRIPTION

Unless otherwise indicated, the terms “%” or “percent” when used in connection with an ingredient of the toothpaste compositions of the invention is intended to refer to the percent by weight of the indicated ingredient in the toothpaste composition.

As used herein, “cleaning” generally refers to the removal of contaminants, dirt, impurities, and/or extraneous matter on a target surface. For example, in the context of oral surfaces, where the surface is tooth enamel, the cleaning may remove at least some of a film or stain, such as plaque biofilm, pellicle or tartar.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, and unless specified otherwise, the term “effective amount” refers to an amount that can promote the growth of beneficial endogenous bacteria in the oral cavity and inhibit pathogenic oral bacteria and/or decrease virulence gene expression and reduce the inflammatory response of oral keratinocytes.

Saccharide prebiotics for use in the present invention are sugars or sugar derivatives, e.g., amide derivatives, amino sugars or sugar alcohols, for example mono-, di- or tri-saccharides (including amino-saccharides and sugar alcohols) which are orally acceptable (i.e., non-toxic at relevant concentrations in an oral care formulation) and which promote the growth of beneficial oral bacteria, while simultaneously negatively affecting the growth of pathogenic oral bacteria. In particular embodiments, the saccharide prebiotic comprises mannose (e.g., D-mannose) (e.g., D-(+)-mannose) and/or N-Acetyl-D-mannosamine.

“D-mannose” is a monosaccharide which is a sugar monomer of the aldohexose series of carbohydrates. It is a C-2 epimer of glucose. Mannose commonly exists as two different-sized rings, the pyranose (six-membered) form and the furanose (five-membered) form. Each ring closure can have either an alpha or beta configuration at the anomeric position. “D-mannose” and “D-(+)-mannose” can be used interchangeably herein.

“N-Acetyl-D-mannosamine” (NADM) is a hexosamine monosaccharide. It is a neutral, stable naturally occurring compound. N-Acetylmannosamine is also known as N-Acetyl-D-mannosamine monohydrate, (which has the CAS Registry Number: 676347-48-1).

In some aspects, the oral care compositions disclosed herein, e.g., any of Composition 1, et seq., comprise water. Water employed in the preparation of the oral care compositions disclosed herein, e.g., any of Composition 1, et seq., should be deionized and free of organic impurities. Water may make up the balance of the oral care composition. In some embodiments, the oral care compositions disclosed herein, e.g., any of Composition 1, et seq., comprise 0 to 90 weight % water, e.g., 0.1 to 90 weight % water, e.g., 1 to 80 weight % water, e.g., 2 to 70 weight % water, 5 to 60 weight % water, e.g., 5 to 50 weight % water, e.g., 20 to 60 weight % water, e.g., 10 to 40 weight % water. This amount of water includes the free water that is added plus that amount which is introduced with other components of the oral care composition, such as with sorbitol.

A thickener provides a desirable consistency and/or stabilizes and/or enhances performance (e.g., provides desirable active release characteristics upon use) of the oral care composition. In some embodiments, the oral care compositions disclosed herein, e.g., any of Composition 1, et seq., comprise from 0.01 to 15 weight % of a thickener, 0.1 to 15 weight % of a thickener, e.g., 0.1 to 10 weight % of a thickener, e.g., 0.1 to 5 weight % of a thickener, e.g., 0.5 to 10 weight % of a thickener, e.g., 0.5 to 5 weight % of at a thickener, e.g., 1 to 4 weight % of a thickener, e.g., 2 to 5 weight % of a thickener, e.g., 2 to 4 weight % of a thickener, e.g., 3 to 4 weight % of a thickener. Higher weight percentages may be used for chewing gums, lozenges and breath mints, sachets, non-abrasive gels and subgingival gels. Thickeners that may be used in the oral care compositions disclosed herein, e.g., any of Composition 1, et seq., include, for example, carboxyvinyl polymers, carrageenan (also known as carrageenan gum), hydroxyethyl cellulose (HEC), natural and synthetic clays (e.g., Veegum and laponite), water soluble salts of cellulose ethers (e.g., sodium carboxymethylcellulose (CMC) and sodium carboxymethyl hydroxyethyl cellulose), natural gums (e.g., gum karaya, xanthan gum, gum arabic, and gum tragacanth), colloidal magnesium aluminum silicate, silica (e.g., finely divided silica), cross-linked poly(vinyl)pyrrolidone, carbowaxes, fatty acids and salts thereof (e.g., stearic acid and palmitic acid), fatty alcohols (e.g., stearyl alcohol), and mixtures thereof. In some embodiments, a mixture of thickening silica and carrageenan gum is used as the thickener in the oral care compositions disclosed herein, e.g., any of Composition 1, et seq. In some embodiments, the oral care compositions disclosed herein, e.g., any of Composition 1, et seq., comprise from 0.01 to 15 weight % of thickening silica and carrageenan gum, 0.1 to 15 weight % of thickening silica and carrageenan gum, e.g., 0.1 to 10 weight % of thickening silica and carrageenan gum, e.g., 0.1 to 5 weight % of thickening silica and carrageenan gum, e.g., 0.5 to 10 weight % of thickening silica and carrageenan gum, e.g., 0.5 to 5 weight % of thickening silica and carrageenan gum, e.g., 1 to 4 weight % of thickening silica and carrageenan gum, e.g., 2 to 5 weight % of thickening silica and carrageenan gum, e.g., 2 to 4 weight % of thickening silica and carrageenan gum, e.g., 3 to 4 weight % of thickening silica and carrageenan gum.

A buffer adjusts the pH of oral care compositions, for example, to a range of about pH 4.0 to about pH 9.5. In some embodiments, the oral care compositions disclosed herein, e.g., any of Composition 1, et seq., comprise from 0.1 to 10 weight % of a buffer, 0.5 to 10 weight % of a buffer, e.g., 0.5 to 5 weight % of a buffer, e.g., 0.5 to 4 weight % of a buffer, e.g., 0.5 to 3 weight % of a buffer, e.g., 0.5 to 2 weight % of a buffer, e.g., 1 to 2 weight % of a buffer. Buffers that may be used in the oral care compositions disclosed herein, e.g., any of Composition 1, et seq., include, for example, sodium bicarbonate, sodium phosphate {e.g., monosodium phosphate (NaH2PO4), disodium phosphate (Na2HPO4), trisodium phosphate (Na3PO4)}, sodium hydroxide, sodium carbonate, sodium acid pyrophosphate, citric acid, sodium citrate, and mixtures thereof. In some embodiments, sodium hydroxide is used as the buffer in the oral care compositions disclosed herein, e.g., any of Composition 1, et seq. In some embodiments, the oral care compositions disclosed herein, e.g., any of Composition 1, et seq., comprise from 0.1 to 10 weight % of sodium hydroxide, e.g., 0.5 to 10 weight % of sodium hydroxide, e.g., 0.5 to 5 weight % of sodium hydroxide, e.g., 0.5 to 4 weight % of sodium hydroxide, e.g., 0.5 to 3 weight % of sodium hydroxide, e.g., 0.5 to 2 weight % of sodium hydroxide, e.g., 1 to 2 weight % of sodium hydroxide.

A humectant keeps oral care compositions from hardening upon exposure to air. Certain humectants can also impart desirable sweetness or flavor to oral care compositions. In some embodiments, the oral care compositions disclosed herein, e.g., any of Composition 1, et seq., comprise, on a pure humectant basis, from 0 to 70 weight % of a humectant, e.g., 10 to 70 weight % of a humectant, e.g., 10 to 65 weight % of a humectant, e.g., 10 to 60 weight % of a humectant, e.g., 10 to 50 weight % of a humectant, e.g., 20 to 50 weight % of at a humectant, e.g., 20 to 40 weight % of a humectant. Humectants that may be used in the oral care compositions disclosed herein, e.g., any of Composition 1, et seq., include, for example, glycerin, sorbitol, xylitol, butylene glycol, polyethylene glycol, propylene glycol, trimethyl glycine, and mixtures thereof. In some embodiments, a mixture of glycerin, sorbitol, and propylene glycol is used as the humectant in the oral care compositions disclosed herein, e.g., any of Composition 1, et seq. In some embodiments, the oral care compositions disclosed herein, e.g., any of Composition 1, et seq., comprise, on a pure humectant basis, from 0 to 70 weight % of glycerin, sorbitol, and propylene glycol, e.g., 10 to 70 weight % of glycerin, sorbitol, and propylene glycol, e.g., 10 to 65 weight % of glycerin, sorbitol, and propylene glycol, e.g., 10 to 60 weight % of glycerin, sorbitol, and propylene glycol, e.g., 10 to 50 weight % of glycerin, sorbitol, and propylene glycol, e.g., 20 to 50 weight % of glycerin, sorbitol, and propylene glycol, e.g., 20 to 40 weight % of glycerin, sorbitol, and propylene glycol.

In some embodiments, the oral care compositions disclosed herein, e.g., any of Composition 1, et seq., comprise a surfactant, e.g., selected from anionic, cationic, zwitterionic, and nonionic surfactants, and mixtures thereof. In some embodiments, the surfactant is reasonably stable throughout a wide pH range. In some embodiments, the oral care compositions disclosed herein, e.g., any of Composition 1, et seq., comprise from 0.01 to 10 weight % of a surfactant, e.g., 0.05 to 5 weight % of a surfactant, e.g., 0.1 to 10 weight % of a surfactant, e.g., 0.1 to 5 weight % of a surfactant, e.g., 0.1 to 2 weight % of a surfactant, e.g., 0.5 to 2 weight % of a surfactant. In some embodiments, the oral care compositions disclosed herein, e.g., any of Composition 1, et seq., comprise from 0.01 to 10 weight % of an anionic surfactant, e.g., 0.05 to 5 weight % of an anionic surfactant, e.g., 0.1 to 10 weight % of an anionic surfactant, e.g., 0.1 to 5 weight % of an anionic surfactant, e.g., 0.1 to 2 weight % of an anionic surfactant, e.g., 0.5 to 2 weight % of an anionic surfactant, e.g., 1.5 weight % of an anionic surfactant.

Anionic surfactants that may be used in the oral care compositions disclosed herein, e.g., any of Composition 1, et seq., include, for example,
i. water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomonoglyceride sulfate,
ii. higher alkyl sulfates, such as sodium lauryl sulfate,
iii. higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate, CH₃(CH₂)₁₀CH₂(OCH₂CH₂)₂₀S O₃Na,
iv. higher alkyl aryl sulfonates such as sodium dodecyl benzene sulfonate (sodium lauryl benzene sulfonate), and
v. higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate.

As used herein, “higher alkyl” refers to C6-30 alkyl.

In some embodiments, the oral care compositions disclosed herein, e.g., any of Composition 1, et seq., comprise an anionic surfactant. In some embodiments, the anionic surfactant is the water soluble salt of alkyl sulfates having from 10 to 18 carbon atoms in the alkyl radical and water soluble salts of sulfonated monoglycerides of fatty acids having from 10 to 18 carbon atoms. Sodium lauryl sulfate, sodium lauroyl sarcosinate, and sodium coconut monoglyceride sulfonates are examples of anionic surfactants of that type. In some embodiments, the oral care compositions disclosed herein, e.g., any of Composition 1, et seq., comprise sodium lauryl sulfate, sodium ether lauryl sulfate, or a mixture thereof. In some embodiments, the oral care compositions disclosed herein, e.g., any of Composition 1, et seq., comprise sodium lauryl sulfate. In some embodiments, the oral care compositions disclosed herein, e.g., any of Composition 1, et seq., comprise from 0.01 to 10 weight % sodium lauryl sulfate, e.g., 0.05 to 5 weight % sodium lauryl sulfate, e.g., 0.1 to 10 weight % sodium lauryl sulfate, e.g., 0.1 to 5 weight %, e.g., sodium lauryl sulfate, e.g., 0.1 to 2 weight % sodium lauryl sulfate, e.g., 0.5 to 2 weight % sodium lauryl sulfate, e.g., 1.5 weight % sodium lauryl sulfate.

An abrasive removes debris and surface stains. In some embodiments, the oral care compositions disclosed herein, e.g., any of Composition 1, et seq., comprise 5 to 70 weight % of an abrasive, e.g., 5 to 60 weight % of an abrasive, e.g., 5 to 50 weight % of an abrasive, e.g., 5 to 40 weight % of an abrasive, e.g., 5 to 30 weight % of an abrasive, e.g., 10 to 30 weight % of an abrasive, e.g., 10 to 20 weight % of an abrasive.

Abrasives that may be used in the oral care compositions disclosed herein, e.g., any of Composition 1, et seq., include, for example, a calcium phosphate abrasive, e.g., tricalcium phosphate (Ca₃(PO₄)₂), hydroxyapatite (Ca₁₀(PO₄)₆(OH)₂), dicalcium phosphate dihydrate (CaHPO₄.2H₂O, also sometimes referred to herein as DiCal), calcium pyrophosphate, and mixtures thereof. Calcium carbonate, e.g., precipitated calcium carbonate, may also be employed as an abrasive.

Other abrasives that may be used in the oral care compositions disclosed herein, e.g., any of Composition 1, et seq., include, for example, silica abrasives such as precipitated silicas having a mean particle size of up to about 20 microns, such as Zeodent 115®, marketed by J. M. Huber, as well as sodium metaphosphate, potassium metaphosphate, aluminum silicate, calcined alumina, bentonite or other siliceous materials, or mixtures thereof. Silica abrasives used herein, as well as the other abrasives, may have an average particle size ranging between about 0.1 and about 30 microns, e.g., between about 5 and about 15 microns. The silica abrasives may be from precipitated silica or silica gels, such as silica xerogels. Particular silica xerogels are marketed under the trade name Syloid® by the W. R. Grace & Co. Davison Chemical Division. Precipitated silica materials include those marketed by the J. M. Huber Corp. under the trade name Zeodent®, including the silica carrying the designation Zeodent 115 and 119.

In some embodiments, abrasives that may be used in the oral care compositions disclosed herein, e.g., any of Composition 1, et seq., include silica gels and precipitated amorphous silica having an oil absorption value of about less than about 100 cc/100 g silica and in the range of about 45 cc/100 g to about 70 cc/100 g silica. Oil absorption values are measured using the ASTA Rub-Out Method D281. In some embodiments, the silica comprises colloidal particles having an average particle size of about 3 microns to about 12 microns, and about 5 to about 10 microns.

In some embodiments, the abrasive comprises a large fraction of very small particles, e.g., having a d50 less than about 5 microns, e.g., small particle silica (SPS) having a d50 of about 3 to about 4 microns, e.g., Sorbosil AC AC43® (Ineos). Such small particles may be used in formulations targeted at reducing hypersensitivity. The small particle component may be present in combination with a second larger particle abrasive.

Low oil absorption silica abrasives that may be used in the oral care compositions disclosed herein, e.g., any of Composition 1, et seq., are marketed under the trade designation Sylodent WXA® by Davison Chemical Division of W.R. Grace & Co., Baltimore, Md. 21203. Sylodent 650 XWA®, a silica hydrogel composed of particles of colloidal silica having a water content of about 29% by weight averaging about 7 to about 10 microns in diameter, and an oil absorption of less than about 70 cc/100 g of silica is an example of a low oil absorption silica abrasive that may be used in the oral care compositions disclosed herein, e.g., Composition 1, et seq.

In some embodiments, the oral care composition disclosed herein, e.g., any of Composition 1, e.g, 1.1-1.40, comprise a high cleaning silica. In some embodiments, the oral care compositions disclosed herein, e.g., Composition 1, et seq., comprise 5 to 70 weight % high cleaning silica, e.g., 5 to 60 weight % high cleaning silica, e.g., 5 to 50 weight % high cleaning silica, e.g., 5 to 40 weight % high cleaning silica, e.g., 5 to 30 weight % high cleaning silica, e.g., 10 to 30 weight % high cleaning silica, e.g., 10 to 20 weight % high cleaning silica.

In some embodiments, the oral care compositions disclosed herein, e.g., any of Composition 1, et seq., comprise a sweetener. In some embodiments, the oral care compositions disclosed herein, e.g., any of Composition 1, et seq., comprise 0.005 to 10 weight % of a sweetener, e.g., 0.01 to 10 weight % of a sweetener, e.g., 0.1 to 10 weight % of a sweetener, e.g., from 0.1 to 5 weight % of a sweetener, e.g., from 0.1 to 3 weight % of a sweetener, e.g., from 0.1 to 1 weight % of a sweetener, e.g., from 0.1 to 0.5 weight % of a sweetener. Sweeteners that may be used in the oral care compositions disclosed herein, e.g., any of Composition 1, et seq., include, for example, sucrose, glucose, saccharin, sucralose, dextrose, levulose, lactose, mannitol, sorbitol, fructose, maltose, xylitol, saccharin salts (e.g., sodium saccharin), thaumatin, aspartame, D-tryptophan, dihydrochalcones, acesulfame, cyclamate salts, and mixtures thereof. In some embodiments, sodium saccharin is used as the sweetener in the oral care compositions disclosed herein, e.g., any of Composition 1, et seq. In some embodiments, the oral care compositions disclosed herein, e.g., any of Composition 1, et seq., comprise 0.005 to 10 weight % sodium saccharin, e.g., 0.01 to 10 weight % sodium saccharin, e.g., 0.1 to 10 weight % sodium saccharin, e.g., from 0.1 to 5 weight % sodium saccharin, e.g., from 0.1 to 3 weight % sodium saccharin, e.g., from 0.1 to 1 weight % sodium saccharin, e.g., from 0.1 to 0.5 weight % sodium saccharin.

In some embodiments, the oral care compositions disclosed herein, e.g., any of Composition 1, et seq., comprise a flavorant. In some embodiments, the oral care compositions disclosed herein, e.g., any of Composition 1, et seq., comprise 0.1 to 5 weight % of a flavorant, e.g., 0.1 to 4 weight % of a flavorant, e.g., 0.1 to 3 weight % of a flavorant, e.g., 0.1 to 2 weight % of a flavorant, e.g., 0.5 to 2 weight % of a flavorant, e.g., 0.6 to 2 weight % of a flavorant, e.g., 0.7 to 2 weight % of a flavorant, e.g., 0.8 to 2 weight % of a flavorant e.g., 0.9 to 2 weight % of a flavorant, e.g., 1 to 2 weight % of a flavorant. Flavorants that may be used in the oral care compositions disclosed herein, e.g., any of Composition 1, et seq., include, for example, essential oils, as well as various flavoring aldehydes, esters, alcohols, and similar materials, as well as menthol, carvone, and anethole, as well as mixtures thereof. Examples of essential oils include oils of spearmint, peppermint, wintergreen, sassafras, clove, sage, eucalyptus, marjoram, cinnamon, lemon, lime, grapefruit, and orange. In some embodiments, a mixture of peppermint oil and spearmint oil is used as the flavorant in the oral care compositions disclosed herein, e.g., any of Composition 1, et seq.

In some embodiments, the oral care compositions disclosed herein, e.g., any of Composition 1, et seq., comprise a pigment. In some embodiments, the oral care compositions disclosed herein, e.g., any of Composition 1, et seq., comprise 0.001 to 20 weight % of a pigment, e.g., 0.01 to 20 weight % of a pigment, e.g., 0.01 to 20 weight % of a pigment, e.g., 0.1 to 20 weight % of a pigment, e.g., 0.1 to 10 weight % of a pigment, e.g., 0.1 to 5 weight % of a pigment, e.g., 0.1 to 3 weight % of a pigment, e.g., 0.1 to 1 weight % of a pigment. In some embodiments, the oral care compositions disclosed herein, e.g., any of Composition 1, et seq., comprise titanium dioxide. In some embodiments, the oral care compositions disclosed herein, e.g., any of Composition 1, et seq., comprise 0.001 to 20 weight % titanium dioxide, e.g., 0.01 to 20 weight % titanium dioxide, e.g., 0.01 to 20 weight % titanium dioxide, e.g., 0.1 to 20 weight % titanium dioxide, e.g., 0.1 to 10 weight % titanium dioxide, e.g., 0.1 to 5 weight % titanium dioxide, e.g., 0.1 to 3 weight % titanium dioxide, e.g., 0.1 to 1 weight % titanium dioxide.

In some embodiments, the oral care compositions disclosed herein, e.g., any of Composition 1, et seq., further comprise an anti-caries agent. In some embodiments, the oral care compositions disclosed herein, e.g., any of Composition 1, et seq., comprise 0.005 to 10 weight % of the anti-caries agent, e.g., 0.01 to 10 weight % of the anti-caries agent, e.g., 0.01 to 5 weight % of the anti-caries agent, e.g., 0.01 to 1 weight % of the anti-caries agent, e.g., 0.01 to 0.3 weight % of the anti-caries agent, e.g., 0.1 to 10 weight % of the anti-caries agent, e.g., 0.1 to 5 weight % of the anti-caries agent, e.g., 0.1 to 2 weight % of the anti-caries agent, e.g., 0.1 to 1 weight % of the anti-caries agent, e.g., 0.1 to 0.8 weight % of the anti-caries agent, e.g., 0.1 to 0.6 weight % of the anti-caries agent, e.g., 0.1 to 0.5 weight % of the anti-caries agent. In some embodiments, the anti-caries agent is a fluoride ion source. In some embodiments, the oral care compositions disclosed herein, e.g., any of Composition 1, et seq., further comprise 0.005 to 10 weight % of the anti-caries agent which is a fluoride ion source, e.g., 0.01 to 10 weight % of the anti-caries agent which is a fluoride ion source, e.g., 0.01 to 5 weight % of the anti-caries agent which is a fluoride ion source, e.g., 0.01 to 1 weight % of the anti-caries agent which is a fluoride ion source, e.g., 0.01 to 0.3 weight % of the anti-caries agent which is a fluoride ion source, e.g., 0.1 to 10 weight % of the anti-caries agent which is a fluoride ion source, e.g., 0.1 to 5 weight % of the anti-caries agent which is a fluoride ion source, e.g., 0.1 to 2 weight % of the anti-caries agent which is a fluoride ion source, e.g., 0.1 to 1 weight % of the anti-caries agent which is a fluoride ion source, e.g., 0.1 to 0.8 weight % of the anti-caries agent which is a fluoride ion source, e.g., 0.1 to 0.6 weight % of the anti-caries agent which is a fluoride ion source, e.g., 0.1 to 0.5 weight % of the anti-caries agent which is a fluoride ion source.

Examples of fluoride ion sources that may be used in the oral compositions disclosed herein, e.g., any of Composition 1, et seq., are found in U.S. Pat. No. 3,535,421 to Briner et al.; U.S. Pat. No. 4,885,155 to Parran, Jr. et al., and U.S. Pat. No. 3,678,154 to Widder et al, incorporated herein by reference in their entirety. Other examples of fluoride ion sources include, for example, stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof. In certain embodiments the fluoride ion source includes stannous fluoride, sodium fluoride, and sodium monofluorophosphate, as well as mixtures thereof. In some embodiments, the anti-caries agent is sodium fluoride. In some embodiments, the oral care compositions disclosed herein, e.g., Composition 1, et seq., comprise 0.005 to 10 weight % sodium fluoride, e.g., 0.01 to 10 weight % sodium fluoride, e.g., 0.01 to 5 weight % sodium fluoride, e.g., 0.01 to 1 weight % sodium fluoride, e.g., 0.01 to 0.3 weight % sodium fluoride, e.g., 0.1 to 10 weight % sodium fluoride, e.g., 0.1 to 5 weight % sodium fluoride, e.g., 0.1 to 2 weight % sodium fluoride, e.g., 0.1 to 1 weight % sodium fluoride, e.g., 0.1 to 0.8 weight % sodium fluoride, e.g., 0.1 to 0.6 weight % sodium fluoride, e.g., 0.1 to 0.5 weight % sodium fluoride.

In some embodiments, the oral care compositions disclosed herein, e.g., any of Composition 1, et seq., comprise the anti-caries agent which is a fluoride ion source in an amount sufficient to supply 25 ppm to 25,000 ppm of fluoride ions, e.g., from 100 to 20,000 ppm of fluoride ions, e.g., from 300 to 15,000 ppm of fluoride ions, e.g., from 500 to 10,000 ppm of fluoride ions, e.g., from 500 to 8,000 ppm of fluoride ions, e.g., from 500 to 6,000 ppm of fluoride ions, e.g., from 500 to 4,000 ppm of fluoride ions, e.g., from 500 to 2,000 ppm of fluoride ions, e.g., from 500 to 1,800 ppm of fluoride ions, e.g., from 1000 to 1600 ppm, e.g., 1450 ppm of fluoride ions. The appropriate level of fluoride ions will depend on the particular application. In some embodiments, toothpaste for consumer use comprises the anti-caries agent which is a fluoride ion source in an amount sufficient to supply from 1,000 to 1,500 ppm of fluoride ions, with pediatric toothpaste having somewhat less. In some embodiments, a dentifrice or coating for professional application comprises the anti-caries agent which is a fluoride ion source in an amount sufficient to supply from 5,000 to 25,000 ppm of fluoride ions.

A whitening agent whitens a tooth to which it is applied. In some embodiments, the oral care compositions disclosed herein, e.g., any of Composition 1, et seq., comprise a whitening agent. In some embodiments, the oral care compositions disclosed herein, e.g., any of Composition 1, et seq., comprise a whitening agent in a dental surface-whitening effective amount, e.g., 0.1 to 90 weight % whitening agent, e.g., 0.5 to 50 weight % whitening agent, e.g., 1 to 30 weight % whitening agent, e.g., 2 to 10 weight % whitening agent. Examples of whitening agents that may be used in the oral compositions disclosed herein, e.g., any of Composition 1, et seq., include, for example, peroxides, metal chlorites, perborates, percarbonates, peroxyacids, hypochlorites, and mixtures thereof. In some embodiments, the whitening agent is hydrogen peroxide or a hydrogen peroxide source, for example, urea peroxide or a peroxide salt or complex (for example, peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or a hydrogen peroxide polymer complex (for example, a peroxide-polyvinyl pyrrolidone polymer complex).

The following examples are further illustrative of the nature of the present invention, but it is understood that the invention is not limited thereto. All amounts and proportions referred to herein and in the appended claims are by weight relative to the total composition, unless otherwise indicated.

### EXAMPLES

### Example 1—Effects of Substrates on Multi-Species Biofilm Composition

Biofilms are grown vertically on Calcium Deficient Hydroxyapatite (CAD-HA) disks (Hitemco Medical, Old Bethpage, USA) using the Amsterdam Active Adhesion model. Biofilms are allowed to establish during 24 h (37° C., 170 rpm) under micro-aerophilic conditions (6% O₂, 7% CO₂, 7% H₂, 80% N₂). After this 24 h, disks are rinsed 3 times a day for 3 minutes (RT, 250 rpm), for 3 consecutive days, by transferring the lid containing the disks to a new 24-well plate containing 2 mL/well of the appropriate substrate solution. As a negative control, PBS (pH 5.7) without substrate supplementation is used. After each rinsing step, disks are shortly dip-rinsed in a new 24-well plate containing fresh modified BHI medium (2 mL/well) to remove remaining substrate traces. Subsequently, the lid is transferred to another 24-well plate containing fresh modified BHI medium (2 mL/well), followed by incubation (micro-aerophilic, 37° C., 170 rpm) until the next rinsing step.

NADM, N-acetyl-D-glucosamine (“NADG”) and D-(+)-mannose are selected to determine their effects on multi-species biofilm composition. Repeated rinsing of the biofilms with the substrates at a concentration of 1M results in a significant decrease in absolute numbers in at least four of the pathogenic bacterial species tested in the assay, compared to the control, the results of which are described in Table 1 below. Absolute abundances of bacterial species are shown as mean±SD (n=3) logarithmic values of the genome equivalents per millilitre (log(Geq/mL)). All substrates are dissolved in PBS at a concentration of 1 M. Statistically significantly different values when compared to the control (PBS) are marked with an asterisk ‘*’ ‘*’ (vs. control (PBS)), ‘#1’ (vs. NADM), ‘#2’ (vs. NADG), ‘#3’ (vs. D-(+)-mannose) (P<0.05):

TABLE 1

| 1M | control | NADM | NADG | D-Mannose |
|---|---|---|---|---|
| Aa | 8.84 ± 0.24^{#2, 3} | 7.84 ± 0.53 | 6.6 ± 0.93* | 6.07 ± 0.47* |
| Fn | 8.92 ± 0.49^{#2, 3} | 8.14 ± 0.44 | 7.09 ± 0.30* | 6.59 ± 0.36* |
| Pg | 6.49 ± 0.80^{#2, 3} | 5.42 ± 0.23 | 4.00 ± 0.39*^{,} ^{#3} | 2.75 ± 0.08*^{, #2} |
| Pi | 8.41 ± 0.42^{#2} | 9.07 ± 0.06 | 6.93 ± 0.62* | 7.80 ± 0.57 |
| S. mutans | 8.57 ± 0.40^{#1} | 7.41 ± 0.44* | 8.95 ± 0.38 | 8.60 ± 0.09 |
| S. sobrinus | 5.91 ± 0.32^{#2} | 6.05 ± 0.23 | 4.16 ± 0.44* | 5.44 ± 0.31 |
| An | 6.74 ± 0.33^{#3} | 6.73 ± 0.39 | 6.52 ± 0.20 | 5.54 ± 0.24* |
| Av | 8.12 ± 0.70^{#3} | 8.35 ± 0.30 | 6.92 ± 0.45 | 6.40 ± 0.48* |
| S. gord. | 8.52 ± 0.27 | 8.94 ± 0.32 | 9.38 ± 0.40 | 9.19 ± 0.39 |
| S. mitis | <LOD | <LOD | <LOD | <LOD |
| S. oralis | 6.96 ± 0.27^{#1, 2} | 9.46 ± 0.24*^{, #2} | 7.97 ± 0.31*^{, #1} | 6.83 ± 0.09 |
| S. sal. | 3.23 ± 0.50 | 3.34 ± 0.55 | 4.38 ± 1.23 | 3.63 ± 0.90 |
| S. sang. | 7.70 ± 0.61 | 9.10 ± 0.29 | 8.11 ± 0.68 | 7.39 ± 0.89 |
| Vp | 10.17 ± 0.33 | 10.76 ± 0.09 | 10.57 ± 0.24 | 10.18 ± 0.09 |

Note:
“Aa”: A. actinomycetemcomitans; “Fn”: F. nucleatum; “Pg”: P. gingivalis; “Pi”: P. intermedia; “An”: A. naeslundii; “Av:” A. viscosus; S. gord.: S. gordonii; S. sal.: S. salivarius; S. sang.: S. sanguinis; Vp: V. parvula; NADG: N-acetyl-D-glucosamine.

In terms of absolute numbers (expressed as the log value of the genome equivalents per millilitre; log(Geq/mL)) and at a substrate concentration of 1 M (Table 1, above), NADM results in a significant decrease (−1.2 log(Geq/mL)) in S. mutans numbers and a significant increase (+2.5 log(Geq/mL)) in S. oralis numbers when compared to the control. NADG results in significant decreases in A. actinomycetemcomitans, F. nucleatum, P. gingivalis, P. intermedia and S. sobrinus numbers (−1.5 to −2.5 log(Geq/mL), and significantly increases S. oralis numbers (+1.0 log(Geq/mL)). D-(+)-mannose yields significantly reduced A. actinomycetemcomitans, F. nucleatum, P. gingivalis, A. naeslundii and A. viscosus numbers (−1.2 to −3.7 log(Geq/mL). When comparing the numbers of pathogens between the different substrate conditions, only for P. gingivalis a significant difference is observed, with D-(+)-mannose resulting in significantly lower amounts compared to NADG (−1.25 log(Geq/mL). For the beneficials/commensals, only for S. oralis is a significant difference obtained, with NADM yielding significantly higher numbers compared to NADG (+1.5 log(Geq/mL)).

In terms of relative biofilm composition (expressed as % (Geq/mL)), the control condition results in a biofilm harbouring 86.39±0.33% beneficials/commensals, 10.68±1.75% periodontal pathogens and 2.93±1.84% cariogenic pathogens (Table 1a). Compared to this, significant increases in beneficials/commensals are observed following treatment with each of the substrates (97.61±0.28% for NADM, 97.52±0.82% for NADG and 96.83±0.62% for D-(+)-mannose). The periodontal pathogens are significantly decreased for the NADM (2.34±0.31%), NADG (0.13±0.10%) and D-(+)-mannose (0.77±0.82%) conditions, whereas the cariogenic pathogens remain unaffected. For the periodontal pathogens there is also a significant decrease in proportion compared to NADM for the NADG (0.13±0.10% vs. 2.34±0.31%) and D-(+)-mannose (0.77±0.82% vs. 2.34±0.31%) conditions.

TABLE 1a

| % | Beneficial or | Periodontal | Cariogenic |
|---|---|---|---|
| (Geq/mL) | commensals | Pathogens | Pathogens |
| Control | 86.39 ± 0.33^{#1, 2, 3} | 10.68 ± 1.75^{#1, 2, 3} | 2.93 ± 1.84 |
| NADM | 97.61 ± 0.28* | 2.34 ± 0.31^{#2, 3} | 0.06 ± 0.04 |
| NADG | 97.52 ± 0.82* | 0.13 ± 0.10 *^{, #1} | 2.35 ± 0.92 |
| D- | 96.83 ± 0.62* | 0.77 ± 0.82 *^{, #1} | 2.40 ± 0.85 |
| mannose | | | |

Relative abundances of the different groups (beneficial/commensals, periodontal pathogens, cariogenic pathogens) of bacterial species are shown as mean±SD (n=3) percentage of the genome equivalents per millilitre (% (Geq/mL)). All substrates are dissolved in PBS at a concentration of 1 M. Statistically significantly different values are marked with ‘*’ (vs. control (PBS)), ‘#1’ (vs. NADM), ‘#2’ (vs. NADG), ‘#3’ (vs. D-(+)-mannose) (P<0.05). Statistically significant values between experimental conditions are only considered to be relevant if each condition is also significantly different from the control condition and only such differences are indicated. NADM: N-acetyl-D-mannosamine; NADG: N-acetyl-D-glucosamine.

When rinsing the biofilms with the substrates at a concentration of 1%_{(w/v)}, At a substrate concentration of 1%_{(w/v)}, only F. nucleatum and S. mutans showed a significant difference with the control, with decreases of 1.1 log(Geq/mL) and 0.5 log(Geq/mL) for D-(+)-mannose and NADM, respectively. No relevant significant differences are observed between substrate conditions. The results are detailed in Table 2:

TABLE 2

| 1%(w/v) | control | NADM | NADG | D-mannose |
|---|---|---|---|---|
| Aa | 9.49 ± 0.25 | 9.51 ± 0.05 | 9.11 ± 0.20 | 9.02 ± 0.11 |
| Fn | 9.71 ± 0.11^{#3} | 9.60 ± 0.22 | 9.41 ± 0.19 | 8.66 ± 0.29 |
| Pg | 7.27 ± 0.20 | 7.06 ± 0.30 | 7.14 ± 0.28 | 7.08 ± 0.17 |
| Pi | 8.02 ± 0.24 | 7.48 ± 0.40 | 8.17 ± 0.22 | 8.06 ± 0.19 |
| S. mutans | 7.48 ± 0.13^{#1} | 7.01 ± 0.05* | 7.83 ± 0.23 | 7.34 ± 0.12 |
| S. | 5.16 ± 0.07 | 5.24 ± 0.27 | 4.87 ± 0.22 | 5.28 ± 0.44 |
| sobrinus | | | | |
| An | 7.58 ± 0.13 | 7.60 ± 0.22 | 7.71 ± 0.12 | 7.59 ± 0.05 |
| Av | 8.01 ± 0.44 | 7.73 ± 0.09 | 7.83 ± 0.31 | 8.00 ± 0.17 |
| S. gord. | 8.33 ± 0.18 | 8.40 ± 0.10 | 8.33 ± 0.06 | 8.29 ± 0.11 |
| S. mitis | < LOD | < LOD | < LOD | 3.00 ± 0.49 |
| S. oralis | 6.90 ± 0.20 | 7.49 ± 0.30 | 7.11 ± 0.14 | 6.27 ± 0.21 |
| S. sal. | 2.86 ± 0.30 | 2.90 ± 0.36 | 2.65 ± 0.00 | 2.89 ± 0.35 |
| S. sang. | 8.37 ± 0.25 | 8.62 ± 0.39 | 8.4 ± 0.11 | 8.19 ± 0.11 |
| Vp | 10.07 ± 0.16 | 10.05 ± 0.17 | 9.77 ± 0.19 | 9.93 ± 0.20 |

Note:
“Aa”: A. actinomycetemcomitans; “Fn”: F. nucleatum; “Pg”: P. gingivalis; “Pi”: P. intermedia; “An”: A. naeslundii; “Av:” A. viscosus; S. gord.: S. gordonii; S. sal.: S. salivarius; S. sang.: S. sanguinis; Vp: V. parvula; “NADG”: N-acetyl-D-glucosamine. Absolute abundances of bacterial species are shown as mean ± SD (n = 3) logarithmic values of the genome equivalents per millilitre (log(Geq/mL)). All substrates are dissolved in PBS at a concentration of 1%(w/v). Statistically significantly different values are marked with
*(vs. control (PBS)),
^{#1}(vs. NADM), ‘#2’ (vs. NADG),
^{#3}(vs. D-(+)-mannose) (P < 0.05).
Statistically significantly different values between experimental conditions are only considered to be relevant if each condition is also significantly different from the control condition and only such differences are indicated.

In the same experiment that produced the data described in Table 2, in terms of relative biofilm composition, the control condition yields a biofilm consisting of 59.22±1.17% beneficials/commensals, 40.63±1.13% periodontal pathogens and 0.16±0.06% cariogenic pathogens. Compared to this, D-(+)-mannose results in a significant increase in beneficials/commensals (84.18±2.04%) and a significant decrease in periodontal pathogens (15.61±2.00%). Furthermore, NADG yields a significant increase in cariogenic pathogens (0.66±0.27%). No relevant significant differences are observed between substrate conditions. These results are detailed in Table 3:

TABLE 3

| % | | Periodontal | Cariogenic |
|---|---|---|---|
| (Geq/mL) | Beneficials/Commensals | Pathogens | Pathogens |
| Control | 59.22 ± 1.17^{#3} | 40.63 ± 1.13^{#3} | 0.16 ± 0.06^{#2} |
| NADM | 61.62 ± 4.88 | 38.32 ± 4.86 | 0.05 ± 0.01 |
| NADG | 60.07 ± 8.25 | 39.27 ± 7.98 | 0.66 ± 0.27* |
| D- | 84.18 ± 2.04 | 15.61 ± 2.00* | 0.21 ± 0.04 |
| mannose | | | |

Relative abundances of the different groups (beneficial/commensals, periodontal pathogens, cariogenic pathogens) of bacterial species are shown as mean ± SD (n = 3) percentage of the genome equivalents per millilitre (%(Geq/mL)). All substrates are dissolved in PBS at a concentration of 1%(w/v). Statistically significantly different values are marked with
*(vs. control (PBS)), ‘#1’ (vs. NADM),
^{#2}(vs. NADG),
^{#3}(vs. D-(+)-mannose) (P < 0.05).
Statistically significantly values between experimental conditions are only considered to be relevant if each condition is also significantly different from the control condition and only such differences are indicated. NADM: N-acetyl-D-mannosamine; NADG: N-acetyl-D-glucosamine.

### Example 2—Effects of Substrates on Multi-Species Biofilm Organic Acid Balances

To examine the effect of NADM and the influence of the orientation and absence/presence of the N-acetyl group on organic acid production and consumption, supernatant samples from substrate-treated or untreated multi-species biofilms are analysed. Significant differences between two substrate conditions are only considered relevant if they are each also significantly different from the control condition. At a substrate concentration of 1 M and when compared to the control, lactate production is unaffected whereas formate production significantly increases for NADM (791.9±138.27 vs. 384.42±19.24 mg/L). Acetate production significantly decreases for NADM and D-(+)-mannose (3084±174 and 1934±56 mg/L, respectively, vs. 3779±305 mg/L). Significantly increases propionate production is observed for NADG and D-(+)-mannose (2750±40 and 2821±127 mg/L, respectively, vs. 2094±132 mg/L). Finally, butyrate production significantly decreases for NADM, NADG and D-(+)-mannose (541±52, 1255±51 and 85±15 mg/L, respectively, vs. 1870±93 mg/L). When comparing relevant significant differences between substrate conditions, an increase in acetate production is observed for NADM compared to D-(+)-mannose (3084±174 vs. 1934±56 mg/L). Butyrate production is significantly lower for NADM and D-(+)-mannose compared to NADG (541±52 and 85±15 mg/L vs. 1255±51), with in addition D-(+)-mannose yielding lower butyrate production in comparison with NADM (541±52 vs. 85±15 mg/L). The results are demonstrated in Table 4 below.

Rinsing with substrates at concentrations of 1%_{(w/v)} did not result in significant differences in organic acid production/consumption compared to the control as also demonstrated in Table 4:

TABLE 4

| | | | |
|---|---|---|---|
| control | NADM | NADG | D-mannose |

1M

| | | | | |
|---|---|---|---|---|
| lactate | −125 ± 0 | 125 ± 0 | 125 ± 0 | 125 ± 0 |
| formate | 384 ± 19^{#1} | 792 ± 138* | 352 ± 18 | 398 ± 12 |
| acetate | 3779 ± 305^{#1, 3} | 3084 ± 174*^{, #3} | 3815 ± 77 | 1934 ± 56*^{, #1} |
| propionate | 2094 ± 132^{#2, 3} | 2052 ± 54 | 2750 ± 40* | 2821 ± 127* |
| butyrate | 1870 ± 93^{#1, 2, 3} | 541 ± 52*^{, #2, 3} | 1255 ± 51*^{, #1, 3} | 86 ± 15*^{, #1, 2} |

1%(w/v)

| | | | | |
|---|---|---|---|---|
| lactate | −125 ± 0 | 125 ± 0 | 125 ± 0 | 125 ± 0 |
| acetate | 345 ± 63 | 396 ± 11 | 379 ± 10 | 352 ± 24 |
| formate | 3954 ± 539 | 3768 ± 174 | 4315 ± 198 | 3717 ± 114 |
| propionate | 2077 ± 234 | 1891 ± 79 | 2205 ± 89 | 1962 ± 28 |
| butyrate | 2129 ± 240 | 1989 ± 44 | 2238 ± 59 | 1985 ± 37 |

Organic acid levels detected in the supernatants of substrate-treated multi-species biofilms are shown as mean ± SD (n = 3) values (mg/L). Values > 0 mg/L represent organic acid production, values <0 mg/L represent organic acid consumption. Substrates are dissolved in PBS at a concentration of 1M (upper part) or 1%(w/v) (lower part). Statistically significantly different values are marked with
*(vs. control (PBS)),
^{#1}(vs. NADM),
^{#2}(vs. NADG),
^{#3}(vs. D-(+)-mannose) (P < 0.05).
Statistically significantly different values between experimental conditions are only considered to be relevant if each condition is also significantly different from the control condition and only such differences are indicated. NADM: N-acetyl-D-mannosamine; NADG: N- acetyl-D-glucosamine.

### Example 3—Effects of Substrates on Multi-Species Biofilm Virulence Gene Expression

The expression of 33 virulence genes from 4 periodontal pathogens is analysed to evaluate the relative virulence of the substrate-treated biofilms. For all three substrates, virulence gene expression is determined relatively to the control, and these values are also compared between substrates when this is relevant (Tables 5a-5d). Significantly different gene expressions in the substrate-treated biofilms relatively to the untreated biofilms are considered to be biologically relevant if their value is more than 2-fold downregulated or more than 1.5-fold upregulated. Only these results are considered. Furthermore, significant differences between two substrate conditions are only considered relevant if they are each also significantly different from the control condition.

At a substrate concentration of 1 M, A. actinomycetemcomitans and P. gingivalis virulence gene expression is generally significantly downregulated relative to the control for all three substrates (2- to 100-fold for A. actinomycetemcomitans and 2.3- to 14.3-fold for P. gingvalis) (Tables 5a-5b). Noteworthy is the significant upregulation of A. actinomycetemcomitans expression of flp for the NADG condition (2.4-fold) and pgA for all 3 substrate conditions (3.5-11.6-fold). In contrast with all this, F. nucleatum virulence gene expression is significantly upregulated for all 3 substrates for the ABC transporter permease gene and the hemin receptor gene (2.5 to 3.8-fold), for NADG and D-(+)-mannose for the hemolysin gene (1.9- and 2.1-fold) and for D-(+)-mannose for the butyrate acetoacetate CoA transferase gene (2.3-fold) (Table 5c).

For P. intermedia, a more diverse effect on virulence gene expression is observed, with in general significant decreases for NADM and D-(+)-mannose (2.5- to 33.3-fold) and increases for NADG (2- to 20.3-fold) (Table 5d). When only considering relevant significant differences between substrates, NADM usually yields higher A. actinomycetemcomitans gene expressions compared to NADG (Table 5a). The opposite is observed for flp and pgA. D-(+)-mannose results in higher omp100 expression compared to NADM and NADG, whereas the opposite is observed for orf859. cdtB expression is higher for D-(+)-mannose compared to NADG, whereas for pgA this is the opposite. Differences are limited for P. gingivalis, with NADG having lower kgp expression compared to D-(+)-mannose, and D-(+)-mannose having lower rgpA expression compared to NADM and NADG (Table 5b). For F. nucleatum, no relevant significant differences between substrate conditions are observed (Table 5c). For P. intermedia, expression of most genes is significantly higher for NADG when compared to NADM and D-(+)-mannose. clpB, dnaJ and phg expression is lower for D-(+)-mannose in comparison with NADM (Table 5d).

At a substrate concentration of 1%_{(w/v)}, A. actinomycetemcomitans gene expression is significantly decreased relatively to the control for NADM in the case of flp, emaA and omp100 (2.4- to 2.9-fold), for D-(+)-mannose in the case of cdtB, emaA, ltxA, omp100 and vapA (2.4- to 5.9-fold), whereas for NADG it increases in the case of aae and orf859 (1.5- and 2-fold) and decreased in the case of vppA (3.6-fold) (Table 5a). For P. gingivalis and F. nucelatum, the observed pattern of significant differences in gene expression relatively to the control is generally opposed to the one observed for a substrate concentration of 1 M (Tables 5b and 5c). When significantly affected, F. nucleatum gene expression decreases (2.6 to 10-fold) and P. gingivalis gene expression increases (1.8- to 3.5-fold), except for partC expression which is decreased (2.8-fold) for NADG. Relative P. intermedia virulence gene expression is generally downregulated for NADM (2.5- to 7.7-fold), unaffected for NADG and downregulated for D-(+)-mannose for 3 genes (2- to 2.7-fold) (Table 5d).

When only considering relevant significant differences in relative gene expressions between substrates, no differences are observed for A. actinomycetemcomitans, P. gingivalis and F. nucleatum (Tables 5a, 5b, and 5c). For P. intermedia, phg expression is lower for NADM compared to D-(+)-mannose (Table 5d).

Tables 5a-5d:

TABLE 5a
Fold Change in virulence gene expression as measured by SYBR RT-qPCR**

| Genes | NADM 1M | | NADG 1M | | D-mannose 1M | |
|---|---|---|---|---|---|---|
| flp | 0.85 | (0.26-2.82) | 2.40 | (0.67-8.67)* | 1.57 | (0.45-5.41) |
| aae | 0.27 | (0.24-0.31)* | 0.24 | (0.18-0.33)* | 0.16 | (0.06-0.48)* |
| apaH | 0.25 | (0.12-0.54)*_{, #2} | 0.08 | (0.02-0.29)*_{, #1} | 0.12 | (0.02-0.76)* |
| cdtB | 0.31 | (0.12-0.78)*_{, #2} | 0.04 | (0.01-0.20)*_{, #1, 3} | 0.13 | (0.04-0.39)*_{, #2} |
| emaA | 0.36 | (0.17-0.77) | 0.29 | (0.07-1.20) | 0.12 | (0.02-0.69)* |
| ltxA | 0.01 | (0.00-0.01)* | 0.01 | (0.00-0.20)* | 0.02 | (0.01-0.04)* |
| omp100 | 0.04 | (0.03-0.04)*_{, #3} | 0.03 | (0.01-0.05)*_{, #3} | 0.26 | (0.11-0.60)*_{, #1, 2} |
| omp29 | 0.48 | (0.19-1.23) | 0.19 | (0.09-0.38)* | 0.27 | (0.07-1.02)* |
| orf859 | 0.11 | (0.03-0.38)*_{, #3} | 0.13 | (0.05-0.36)*_{, #3} | 0.03 | (0.01-0.09)*_{, #1, 2} |
| pgA | 3.53 | (1.73-7.22)*_{, #2} | 11.60 | (3.23-41.74)*_{, #1, 3} | 5.70 | (3.02-10.73)*_{, #2} |
| vapA | 0.74 | (0.39-1.42) | 1.00 | (0.58-1.75) | 0.62 | (0.25-1.53) |
| vppA | 0.81 | (0.35-1.84) | 0.49 | (0.41-0.59)* | 0.58 | (0.38-0.90) |
| Genes | NADM 1% (w/v) | | NADG 1% (w/v) | | D-mannose 1% (w/v) | |
| flp | 0.35 | (0.11-1.17)* | 0.54 | (0.19-1.56) | 1.26 | (0.84-1.91) |
| aae | 0.92 | (0.72-1.19) | 1.55 | (1.18-2.03)* | 1.06 | (0.52-2.14) |
| apaH | 0.81 | (0.62-1.05) | 0.73 | (0.41-1.29) | 0.72 | (0.38-1.36) |
| cdtB | 0.59 | (0.47-0.74) | 1.05 | (0.61-1.81) | 0.36 | (0.11-1.20)* |
| emaA | 0.40 | (0.16-0.98)* | 0.68 | (0.15-3.07) | 0.41 | (0.14-1.22)* |
| ltxA | 0.29 | (0.03-2.55) | 0.40 | (0.11-1.39) | 0.17 | (0.02-1.28)* |
| omp100 | 0.42 | (0.21-0.81)* | 0.71 | (0.42-1.18) | 0.34 | (0.09-1.23)* |
| omp29 | 0.76 | (0.22-2.57) | 0.77 | (0.69-0.87) | 0.65 | (0.33-1.28) |
| orf859 | 0.57 | (0.28-1.16) | 1.99 | (0.94-4.22)* | 1.46 | (0.8-2.68) |
| pgA | 1.07 | (0.29-3.97) | 0.72 | (0.41-1.27) | 0.55 | (0.41-0.74) |
| vapA | 0.82 | (0.65-1.04) | 0.65 | (0.48-0.86) | 0.39 | (0.11-1.39)* |
| vppA | 1.01 | (0.42-2.41) | 0.28 | (0.09-0.86)* | 0.53 | (0.2-1.4) |

** Results are listed as fold changes relative to the control conditions (2^{\hat{ }-ΔΔCt} method), the values for the control are normalized to “1”. Fold changes in virulence gene expression are determined relative to the control condition through the 2-ΔΔCt method and are shown as the geometric mean (C.I.) (n = 3) of the 2-ΔΔCt values. Statistically significantly fold changes relative to the control (PBS) that are <0.5 (more than 2-fold downregulated) or >1.5 (more than 1.5-fold upregulated) are considered biologically relevant and are marked with h
*(P < 0.05). For such values, statistically significant differences between two experimental conditions are marked with
_{#1}(vs. NADM),
_{#2}(vs. NADG),
_{#3}(vs. D-(+)-mannose) (P < 0.05).

TABLE 5b
Fold Change in virulence gene expression as measured by SYBR RT-qPCR

| | NADM 1M | | NADG 1M | | D-mannose 1M | |
|---|---|---|---|---|---|---|
| kgp | 0.82 | (0.22-3.08) | 0.07 | (0.04-0.13)*_{, #3} | 0.44 | (0.19-1.06)*_{, #2} |
| fimA | 0.50 | (0.11-2.37) | 0.79 | (0.44-1.39) | 1.17 | (0.23-5.86) |
| partC | 0.20 | (0.11-0.37)* | 0.19 | (0.13-0.28)* | 0.09 | (0.02-0.39)* |
| rgpA | 0.43 | (0.25-0.75)*_{, #3} | 0.39 | (0.18-0.84)*_{, #3} | 0.18 | (0.11-0.29)*_{, #1, 2} |
| Genes | NADM 1% | | NADG 1% | | D-mannose 1% | |
| kgp | 3.46 | (1.52-7.91)* | 1.81 | (0.44-7.50) | 3.07 | (0.79-11.87)* |
| fimA | 1.07 | (0.79-1.45) | 1.15 | (0.32-4.06) | 2.50 | (0.69-9.08)* |
| partC | 0.62 | (0.33-1.16) | 0.36 | (0.08-1.68)* | 0.60 | (0.22-1.64) |
| rgpA | 0.88 | (0.52-1.51) | 1.31 | (1.03-1.66) | 1.85 | (0.91-3.76)* |

Statistically significantly fold changes relative to the control (PBS) that are <0.5 (more than 2-fold downregulated) or >1.5 (more than 1.5-fold upregulated) are considered biologically relevant and are marked with
*(P < 0.05). For such values, statistically significant differences between two experimental conditions are marked with
_{#1}(vs. NADM),
_{#2}(vs. NADG),
_{#3}(vs. D-(+)-mannose) (P < 0.05).
Fold changes in virulence gene expression are determined relatively to the control condition through the 2-ΔΔCt method and are shown as the geometric mean (C.I.) (n = 3) of the 2-ΔΔCt values. The data are shown as the geometric mean (C.I.) (C.I.: 95% confidence interval) (n = 3) of the 2^{-ΔΔCt} values.

TABLE 5c
Fold Change in virulence gene expression as measured by SYBR RT-qPCR**

| GENES | NADM 1M | | NADG 1M | | D-mannose 1M | |
|---|---|---|---|---|---|---|
| but.-coA transf. | 0.76 | (0.52-1.13) | 1.21 | (0.99-1.49) | 2.27 | (1.48-3.48)* |
| ompA | 1.27 | (0.69-2.35) | 1.33 | (0.78-2.25) | 1.36 | (0.74-2.47) |
| EF-G | 1.37 | (0.83-2.25) | 1.45 | (0.93-2.26) | 1.36 | (0.99-1.88) |
| ABC transp. perm. | 2.46 | (1.36-4.46)* | 3.33 | (2.53-4.39)* | 2.65 | (1.90-3.71)* |
| transposase | 1.03 | (0.55-1.92) | 1.27 | (0.68-2.37) | 1.46 | (0.70-3.05) |
| hemolysin | 1.05 | (0.72-1.53) | 1.90 | (1.29-2.79)* | 2.14 | (1.37-3.34)* |
| hemin receptor | 2.81 | (2.29-3.45)* | 3.26 | (2.24-4.76)* | 3.82 | (2.06-7.08)* |
| GENES | NADM 1% | | NADG 1% | | D-mannose 1% | |
| but.-coA transf. | 0.38 | (0.05-2.72) | 0.21 | (0.11-0.39)* | 0.37 | (0.06-2.14) |
| ompA | 0.75 | (0.21-2.67) | 0.74 | (0.18-2.98) | 1.17 | (0.38-3.60) |
| EF-G | 0.11 | (0.02-0.74)* | 0.10 | (0.02-0.45)* | 0.13 | (0.04-0.45)* |
| ABC transp. perm. | 0.39 | (0.12-1.25)* | 0.33 | (0.12-0.87)* | 0.73 | (0.40-1.32) |
| transposase | 0.75 | (0.56-1.00) | 0.98 | (0.18-5.39) | 1.38 | (0.45-4.27) |
| hemolysin | 0.55 | (0.08-3.83) | 0.79 | (0.30-2.04) | 0.89 | (0.32-2.53) |
| hemin receptor | 0.10 | (0.02-0.63)* | 0.19 | (0.02-1.67)* | 0.26 | (0.12-0.57)* |

** Results are listed as fold changes relative to the control conditions (2^{\hat{ }-ΔΔCt} method), the values for the control are normalized to “1”. The data are shown as the geometric mean (C.I.) (C.I.: 95% confidence interval) (n = 3) of the 2-^{ΔΔCt} values. Statistically significant fold changes relative to the control (PBS) that are <0.5 (more than 2-fold downregulated) or >1.5 (more than 1.5-fold upregulated) are considered biologically relevant and are marked with
*(P < 0.05). For such values, statistically significant differences between two experimental conditions are marked with ‘#1’ (vs. NADM), ‘#2’ (vs. NADG), ‘#3’ (vs. D-(+)-mannose) (P < 0.05)

TABLE 5d
Fold Change in virulence gene expression as measured by SYBR RT-qPCR**

| Genes | NADM 1M | | NADG 1M | | D-mannose 1M | |
|---|---|---|---|---|---|---|
| adpc | 0.70 | (0.47-1.04) | 2.65 | (1.25-5.62)*, #3 | 0.27 | (0.17-0.41)*, #2 |
| clpB | 0.40 | (0.26-0.61)*, #1, 2 | 2.03 | (0.94-4.41)*, #1, 3 | 0.14 | (0.09-0.24)*, #1, 2 |
| DnaK | 0.70 | (0.46-1.06) | 2.34 | (1.23-4.43)*, #3 | 0.44 | (0.19-1.02)*, #2 |
| DnaJ | 0.15 | (0.08-0.28)*, #3 | 0.64 | (0.35-1.16) | 0.03 | (0.00-0.22)*, #1 |
| ECF | 0.55 | (0.10-3.00) | 20.32 | (4.81-85.88)* | 1.47 | (0.22-9.84) |
| GroES | 0.45 | (0.10-2.04) | 1.86 | (1.38-2.50) | 0.37 | (0.21-0.64)* |
| HtpG | 0.35 | (0.20-0.64)*, #2 | 3.40 | (2.27-5.08)*, #1, 3 | 0.29 | (0.15-0.56)*, #2 |
| KpsD | 0.41 | (0.34-0.51)*,#2 | 1.72 | (0.62-4.72)*, #1, 3 | 0.42 | (0.23-0.77)*, #2 |
| inpA | 0.64 | (0.26-1.58) | 4.52 | (4.14-4.94)*, #3 | 0.47 | (0.13-1.7)*, #2 |
| phg | 0.15 | (0.11-0.21)*, #3 | 0.57 | (0.38-0.85) | 0.10 | (0.06-0.17)*, #1 |
| Genes | NADM 1% | | NADG 1% | | D-mannose 1% | |
| adpc | 0.21 | (0.04-1.06)* | 0.67 | (0.64-0.69) | 0.54 | (0.14-2.03) |
| clpB | 0.40 | (0.07-2.17)* | 1.06 | (0.58-1.96) | 1.00 | (0.21-4.83) |
| DnaK | 0.31 | (0.29-0.32)* | 0.77 | (0.34-1.77) | 0.63 | (0.32-1.22) |
| DnaJ | 0.19 | (0.06-0.65)* | 0.7 | (0.5-0.99) | 0.37 | (0.08-1.7)* |
| ECF | 0.13 | (0.05-0.33)* | 0.48 | (0.11-2.02) | 0.6 | (0.31-1.18) |
| GroES | 0.51 | (0.34-0.76) | 0.75 | (0.49-1.14) | 0.51 | (0.34-0.76) |
| HtpG | 0.21 | (0.04-1.05)* | 0.46 | (0.22-0.94) | 0.46 | (0.24-0.89) |
| KpsD | 0.35 | (0.07-1.69)* | 0.78 | (0.37-1.62) | 0.96 | (0.26-3.47) |
| inpA | 0.28 | (0.11-0.70)* | 0.53 | (0.24-1.17) | 0.37 | (0.15-0.90)* |
| phg | 0.26 | (0.11-0.58)*, #3 | 0.69 | (0.47-1.00) | 0.50 | (0.19-1.28)*, #1 |

** Results are listed as fold changes relative to the control conditions (2^{\hat{ }-ΔΔCt} method), the values for the control are normalized to “1”. The data are shown as the geometric mean (C.I.) (C.I.: 95% confidence interval) (n = 3) of the 2^{-ΔΔCt} values. Statistically significantly different fold changes relative to the control (PBS) that are <0.5 (more than 2-fold downregulated) or >1.5 (more than 1.5-fold upregulated) are considered biologically relevant and are marked with
*(P < 0.05). For such values, statistically significant differences between two experimental conditions are marked with
#1(vs. NADM),
#2(vs. NADG),
#3(vs. D-(+)-mannose) (P < 0.05)

### Example 4—Effects of Substrates on Multi-Species Biofilm Inflammatory Potential

Cultures of immortalized human oral keratinocytes (HOK-18A) are grown. The relative inflammatory potential of the substrate-treated multi-species biofilms is evaluated by analyzing the expression of five inflammatory mediator genes in human oral keratinocytes (HOKs) exposed to the substrate-treated biofilms. Significantly different gene expressions in HOKs exposes the substrate-treated biofilms relatively to HOKs exposed to the control biofilms are considered to be biologically relevant if their value is more than 1.5-fold upregulated or more than 2-fold downregulated. Only these results are considered. The IL-8 levels in the cellular supernatant are determined as well. RNA is converted to cDNA and relative expression of inflammatory mediator genes is determined as described above with respect to the cellular housekeeping gene (3-actin.

In HOKs exposed to substrate-treated (substrate concentrations of 1 M) multi-species biofilms, mostly decreases in inflammatory mediator gene expression are observed and measured via SYBR RT-qPCR, the results of which are detailed in Table 6.

Exposure of HOKs to substrate-treated biofilms (substrate concentration of 1 M) results in decreased IL-8 gene expression (7.1-fold to 10-fold) relative to HOKs exposed to untreated biofilms (Table 6). The relative expression of the other genes is generally unaffected, except for the MMP-8 gene, where it increases for the NADM condition (1.7-fold). When comparing between substrates, no relevant significant differences are observed. Absolute IL-8 levels significantly decrease (36-fold to 357-fold) for all three substrate conditions compared to the control (Table 7). No relevant significant differences in IL-8 levels are observed when comparing between substrates.

Exposure of HOKs to substrate-treated biofilms (substrate concentration of 1%_{(w/v)}) does not generally yield significant differences in gene expression relative to HOKs exposed to untreated biofilms, with the exception of a decreased MMP-8 gene expression for the D-(+)-mannose condition (2.5-fold) (Table 6). No relevant significant differences are observed when comparing between substrates. This is also the case for IL-8 levels when compared to the control condition or between substrates, except for the NADM condition which showed reduced IL-8 levels compared to the control (2.4-fold) (Table 7).

TABLE 6
Effects of repeated rinsing with potentially prebiotic
substrates on multi-species biofilm inflammatory
potential towards human oral keratinocytes.

| Genes | NADM 1M | NADG 1M | D-mannose 1M |
|---|---|---|---|
| IL-1β | 0.59 (0.30-1.14) | 0.75 (0.55-1.02) | 0.81 (0.59-1.12) |
| IL-6 | 0.86 (0.34-2.18) | 0.57 (0.46-0.71) | 0.63 (0.41-0.97) |
| IL-8 | 0.10 (0.05-0.17)* | 0.14 (0.06-0.36)* | 0.12 (0.06-0.27)* |
| MMP-8 | 1.66 (0.82-3.35)* | 1.28 (0.79-2.08) | 1.25 (1.10-1.42) |
| TNF-α | 0.73 (0.64-0.83) | 0.89 (0.59-1.34) | 0.77 (0.37-1.60) |
| Genes | NADM 1% | NADG 1% | D-mannose 1% |
| IL-1β | 0.83 (0.66-1.04) | 0.71 (0.38-1.33) | 0.79 (0.43-1.48) |
| IL-6 | 0.73 (0.48-1.13) | 0.73 (0.58-0.93) | 0.79 (0.60-1.06) |
| IL-8 | 1.32 (1.14-1.53) | 1.00 (0.78-1.27) | 1.22 (0.79-1.86) |
| MMP-8 | 0.62 (0.21-1.84) | 0.52 (0.17-1.60) | 0.40 (0.27-0.58)* |
| TNF-α | 0.69 (0.48-1.00) | 0.71 (0.28-1.35) | 0.770.48-1.23) |

Results are listed as fold changes relative to the control conditions (2^{\hat{ }-ΔΔCt} method), the values for the control are normalized to “1”. The data are shown as the geometric mean (C.I.) (C.I.: 95% confidence interval) (n = 3) of the 2^{-ΔΔCt} values. Statistically significantly different fold changes relative to the control (PBS) that are <0.5 (more than 2-fold downregulated) or >1.5 (more than 1.5-fold upregulated) are considered biologically relevant and are marked with
*(P < 0.05).

As shown in Table 6, fold changes in inflammatory mediator gene expression from human oral keratinocytes (HOK-18A) exposed to substrate-treated multi-species biofilms are determined relative to the control through the 2^{\hat{ }-ΔΔct} method and are shown as the geometric mean (C.I.) (n=3) of the 2^{\hat{ }-ΔΔct} values. All substrates are dissolved in PBS at a concentration of 1 M (upper part) or 1% (w/v) (lower part). Values between 0 and 1 represent relative downregulation, values >1 represent relative upregulation. Statistically significantly fold changes relative to the control (PBS) that are <0.5 (more than 2-fold downregulated) or >1.5 (more than 1.5-fold upregulated) are considered biologically relevant and are marked with ‘*’ (P<0.05). For such values, statistically significant differences between two experimental conditions are marked with ‘#1’ (vs. NADM), ‘#2’ (vs. NADG), ‘#3’ (vs. D-(+)-mannose) (P<0.05).

TABLE 7
Effects of repeated rinsing with potentially prebiotic substrates
on multi-species biofilm inflammatory potential

| | | | |
|---|---|---|---|
| control | NADM | NADG | D-mannose |

1M

| | | | |
|---|---|---|---|
| 21.43 ± 4.67^{#1, 2, 3} | 0.59 ± 0.61* | 0.22 ± 0.32* | 0.06 ± 0.04* |

1%(w/v)

| | | | |
|---|---|---|---|
| 72.52 ± 19.2^{#1} | 30.32 ± 6.26* | 54.75 ± 11.66 | 51.09 ± 13.89 |

Table 7: IL-8 levels detected in the supernatants of human oral keratinocytes (HOK-18A) cultures exposed to substrate-treated multi-species biofilms are shown as mean±SD (n=3) values (pg/mL). All substrates are dissolved in PBS at a concentration of 1 M (upper part) or 1% (w/v) (lower part). Values between 0 and 1 represent relative downregulation, values >1 represent relative upregulation. Statistically significantly fold changes relative to the control (PBS) that are <0.5 (more than 2-fold downregulated) or >1.5 (more than 1.5-fold upregulated) are considered biologically relevant and are marked with (P<0.05). For such values, statistically significant differences between two experimental conditions are marked with ‘#1’ (vs. NADM), ‘#2’ (vs. NADG), ‘#3’ (vs. D-(+)-mannose) (P<0.05).

### Example 5—Compositions Comprising Saccharide Prebiotic

A toothpaste comprising a saccharide prebiotic, e.g., D-mannose and/or N-acetyl-D-mannosamine, is prepared using the following ingredients:

| Ingredient | wt % |
|---|---|
| 70% Sorbitol | 20 |
| Glycerin | 20 |
| Water | Q.S. |
| High Cleaning Silica | 10 |
| Gantrez S-97 | 15 |
| Abrasive Silica | 8.8 |
| Thickening Silica | 2.7 |
| Sodium Lauryl Sulfate | 1.5 |
| Sodium Hydroxide | 0-1.2 |
| Sodium CMC - Type 12 | 1.1 |
| Flavor | 1-1.2 |
| Titanium Dioxide | 0.75 |
| Propylene Glycol | 0.5 |
| Carrageenan Gum | 0.48 |
| Sodium Saccharin | 0.3 |
| Saccharide prebiotic | 1 |
| (e.g., D-mannose and/or | |
| N-acetyl-D-mannosamine) | |

Another toothpaste comprising a saccharide prebiotic is prepared using the following ingredients:

| Ingredient | % |
|---|---|
| 70% Sorbitol | 14 |
| Glycerin | 17 |
| Water | Q.S. |
| High Cleaning Silica | 17 |
| Gantrez S-97 | 17 |
| Thickening Silica | 2.7 |
| Sodium Lauryl Sulfate | 1.5 |
| Sodium Hydroxide | 0-1.2 |
| Sodium CMC - Type 12 | 1.1 |
| Xanthan Gum | 0.8 |
| Flavor | 1-1.2 |
| Titanium Dioxide | 0.5 |
| Propylene Glycol | 0.5 |
| Carrageenan Gum | 0.48 |
| Sodium Saccharin | 0.3 |
| Sodium Fluoride | 0.243 |
| Saccharide prebiotic | 0.5 |
| (e.g, D-mannose and/or | |
| N-acetyl-D-mannosamine) | |

What is claimed is:

1. An oral care composition comprising an effective amount of at least one saccharide prebiotic selected from D-mannose, N-acetyl-D-mannosamine and mixtures thereof in an amount effective to promote the growth of beneficial endogenous bacteria in the oral cavity and inhibit pathogenic oral bacteria.

2. The oral care composition of claim 1, wherein the saccharide prebiotic is D-mannose.

3. The oral care composition of claim 1, wherein the saccharide prebiotic is N-acetyl-D-mannosamine.

4. (canceled)

5. (canceled)

6. The oral care composition of claim 1, wherein the composition promotes the growth or expression in the oral cavity of one or more beneficial endogenous bacterial species, wherein said species are one or more selected from the group consisting of: A. naeslundii; A. viscosus; S. gordonii: S. mitis; S. oralis; S. salivarius; S. sanguinis; V. parvula.

7. The oral care composition of claim 1, wherein the composition negatively affects the growth or expression in the oral cavity of one or more pathogenic bacterial species, wherein said species are one or more selected from the group consisting of: A. actinomycetemcomitans; F. nucleatum; P. gingivalis; P. intermedia; S. mutans; S. Sobrinus.

8. (canceled)

9. The oral care composition of claim 1, wherein the composition further comprises at least one species of bacteria that has beneficial effects on oral health.

10. The oral care composition of claim 9, wherein the species of bacteria that has beneficial effects on oral health is selected from A. naeslundii; A. viscosus; S. gordonii: S. mitis; S. oralis; S. salivarius; S. sanguinis; V. parvula and combinations thereof.

11. The oral care composition of claim 1, wherein the composition is a mouthwash, toothpaste, tooth gel, tooth powder, non-abrasive gel, mousse, foam, mouth spray, lozenge, oral tablet, dental implement.

12. (canceled)

13. The oral care composition of claim 1, wherein the prebiotic substrate can decrease virulence gene expression of one or more of the following genes in A. actinomycetemcomitans: aae, emaA, paH, cdtB, ltxA, omp100, orf859, vapA and flp.

14. The oral care composition of claim 1, wherein the prebiotic substrate can decrease virulence gene expression of one or more of the following genes in P. gingivalis: fim A, kgp, partC, rgpA.

15. The oral care composition of claim 1, wherein the prebiotic substrate can decrease virulence gene expression of one or more of the following genes in P. intermedia: adpc, clpB, DnaK, DnaJ, ECF, GroES, HtpG, KpsD, inpA, phg.

16. The oral care composition of claim 1, wherein the prebiotic substrate can decrease virulence gene expression of one or more of the following genes in F. nucleatum: ABC transporter permease, heroin receptor, EF-G.

17. The oral care composition of claim 1, wherein the composition is in an amount sufficient to decrease the gene expression of one or more inflammatory biomarker in oral keratinocytes.

18. (canceled)

19. A method for selectively promoting, in an oral cavity: growth, metabolic activity or colonization of bacteria that have beneficial effects on oral health, relative to growth, metabolic activity or colonization of pathogenic oral bacteria, comprising administering to the oral cavity an oral care composition comprising a composition of claim 1.

20. A method for selectively promoting, in an oral cavity, biofilm formation by bacteria that have beneficial effects on oral health, relative to biofilm formation by pathogenic oral bacteria, comprising administering to the oral cavity an oral care composition comprising a composition of claim 1.

21. A method for preventing or mitigating one or more of gingivitis, periodontitis, peri-implantitis, peri-implant mucositis, necrotizing gingivitis, necrotizing periodontitis and caries in a subject in need thereof, by selectively promoting in the oral cavity of the subject the growth, metabolic activity or colonization of bacteria that have beneficial effects on oral health, relative to growth, metabolic activity or colonization of pathogenic oral bacteria, comprising administering to the oral cavity an oral care composition comprising a composition of claim 1.

22. (canceled)

23. (canceled)

24.-32. (canceled)
